(19) United States
(12) Patent Application Publication
(10) Pub. No.: US 2024/0294697 A1
(43) Pub. Date: Sep. 5, 2024

(54) AMPHIPHILIC OLIGOMERS AND SELF-ASSEMBLING HYDROGELS FORMED THEREFROM

(71) Applicant: THE GOVERNING COUNCIL OF THE UNIVERSITY OF TORONTO, Toronto (CA)

(72) Inventors: Paul SANTERRE, Toronto (CA); Eli SONE, Toronto (CA); Angus Wen-Tsung LAM, Toronto (CA)

(21) Appl. No.: 18/571,563

(22) PCT Filed: Jun. 20, 2022
(86) PCT No.: PCT/CA2022/050992

§ 371 Date: Dec. 18, 2023

Related U.S. Application Data
(60) Provisional application No. 63/212,459, filed on Jun. 18, 2021.

(51) Int. Cl. C08G 18/42; C08G 18/24; C08G 18/40; C08G 18/48; C08G 18/77

(52) U.S. Cl. CPC C08G 18/4277; C08G 18/246; C08G 18/4018; C08G 18/4833; C08G 18/771; C08G 2210/00; C08G 2230/00

(57) ABSTRACT

Amphiphilic oligomers are provided that yield phase-separated gels suitable for sustained release drug delivery. The amphiphilic backbone includes a hydrophilic domain and a degradable hydrophobic domain, which are linked with an isocyanate group, while the terminal domains on the oligomer are functionalized with catechol adhesive groups capable of cross-linking.

This patent application claims priority to U.S. provisional patent application 63/212,459, filed Jun. 18, 2021, the contents of which are incorporated herein.

### TECHNICAL FIELD

This disclosure generally relates to the field of amphiphilic oligomers and hydrogels.

### BACKGROUND OF THE ART

Localized delivery of therapeutics is of great interest as a means of avoiding systemic effects, particularly those associated with prolonged systemic intake. One challenge associated with localized delivery of therapeutics can be short retention time at the site of administration, which for many applications is via injection, thereby requiring frequent injections. Many types of polymer carriers have been developed for local injections, however, many suffer from one or more of the following shortcomings: poor mechanical properties; not degradable; toxicity of chemical products; poor adhesion locally; and undesirable release profiles. There remains a need for drug delivery systems that mitigate some or all of these shortcomings.

### SUMMARY

In accordance with one aspect, there is provided an amphiphilic oligomer having a formula

(D-C-A-C)ₙ—B or (D-C—B—C)ₙ-A

wherein: n=2 or 3; D is a terminal adhesive group; A is a hydrophilic segment having a molecular weight between 600 and 2000; B is a degradable hydrophobic segment having a molecular weight between 900 and 2000; and C is a linking group comprising a urethane or urea linkage.

In some embodiments, the ratio of hydrophobic segment to hydrophilic segment is between 0.3 and 2.1, preferably between 0.3 and 1.0. In some embodiments, the hydrophobic segment has a molecular weight between 800 and 1400; the hydrophilic segment has a molecular weight between 800 and 1200; and/or, the molecular weight of the amphiphilic oligomer is between 4400 and 10,000.

The hydrophobic segment may be derived from a polyester, including polycaprolactone (PCL), polylactic acid, polyglycolic acid, and polycaprolactone, a polycarbonate, polyamide, polyurethane (PU), cellulosic oligomer, oligosaccharide, poly(alkenedicarboxylate) e.g. poly(butylene succinate), poly(hydroxybutyrate), poly anhydrides, poly peptides, poly(p-hydroxyalcanoate), poly(hydroxybutyrate-co-hydroxyvalerate) or poly(p-dioxanone), preferably polycaprolactone.

The hydrophilic segment may be derived from polyethylene glycol (PEG), polyvinyl alcohol (PVA), polyvinylpyrrolidone (PVP), polyethylene oxide (PEO), co-poly(ethylene oxide)-b-poly(propylene oxide), poly(hydroxyethylmethacrylate) (polyHEMA), poly acrylic acid or poly acrylic acid mono-salts (H⁺ substituted for Na⁺, K⁺), preferably PEG.

The terminal adhesive group is suitably a terminal benzene-1,2-diol derivative or a terminal adhesive benzene-1,2,3-triol derivative, preferably a 4-alkylbenzene-1,2-diol derivative or a 5-alkylbenzene-1,2,3-triol derivative. In some embodiments, D has a structure selected from:

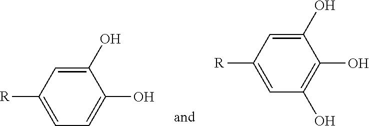

wherein R is present or absent and when present is a C1-C6 alkyl group or C1-C6 alkene optionally substituted with OH, NH₂.

In another aspect, there is provided self-assembled hydrogels comprising one or more oligomers as disclosed herein. A hydrogel may be formed from a single amphiphilic oligomer species, in some embodiments, or a blend of amphiphilic oligomer species in other embodiments.

In one embodiment, there is provided a self-assembled hydrogel formed from a first amphiphilic oligomer of the formula (D-C-A-C)n-B, wherein A is derived from PEG and has a molecular weight of about 1000, B is derived from PCL and has a molecular weight of about 1250 and n=2; and a second amphiphilic oligomer of the formula (D-C-A-C)n-B, wherein the A is derived from PEG and has a molecular weight of about 1000, B is derived from PCL and has a molecular weight of about 900 and n=3; preferably, wherein the self-assembled hydrogel comprises between 40 and 60 wt % of the first amphiphilic oligomer and between 40 and 60 wt % of the second amphiphilic oligomer, based on the combined weight of the amphiphilic oligomers.

In another aspect, there is provided a method of preparing a gel polymer matrix comprising dissolving an amphiphilic oligomer as disclosed herein in an aqueous solvent and raising the temperature of the solution to a gelation temperature of the gel polymer matrix.

In another aspect, there is provided a pharmaceutical composition comprising an amphiphilic oligomer as disclosed herein and an aqueous carrier or a self-assembled hydrogel as disclosed herein. The pharmaceutical composition may include a soluble or non-soluble drug, optionally selected from a small molecule drug, a protein or protein derived peptide, a nucleotide or an oligo nucleotide, in some embodiments, a biomolecule (e.g. VEGF and IL-4) or small molecule pharmaceutical (e.g. an non-steroidal anti-inflammatory drug (NSAID), e.g. celecoxib). In some embodiments, the pharmaceutical composition further includes an antioxidant or hydrogen peroxide scavenger, optionally selected from ascorbic acid, Vitamin E and catalase, preferably catalase. The pharmaceutical composition is suitably injectable at a temperature of between 4° C. and 15° C. or at a temperature of less than 15° C., less than 10° C. or less than 5° C. The pharmaceutical may be in the form of a sustained release drug depot.

In another aspect, there is provided a process of preparing an amphiphilic oligomer comprising:
A) reacting one of a hydrophilic polyol A′ having a molecular weight between 600 and 2000 and a hydrophobic polyol B′ having a molecular weight between 900 and 2000 with a diisocyanate under conditions and for a time sufficient to obtain a compound of the formula

C′-A-C′ or C′—B—C′
wherein A and B are as defined above, and C′ comprises a urethane or urea linkage and an isocyanate group;
B) reacting the reaction product of step A with a hydrophilic polyol having a molecular weight between 600 and 2000 or a hydrophobic polyol having a molecular weight between 900 and 2000;
wherein if a hydrophilic polyol is reacted in step (A), a hydrophobic polyol is reacted in step (B) and if a hydrophobic polyol is reacted in step (A), a hydrophobic polyol is reacted in step (B); and
C) reacting the reaction product of step B with a 4-alkylbenzene-1,2-diol derivative or a 5-alkylbenzene-1,2,3-triol derivative selected from the group consisting of:

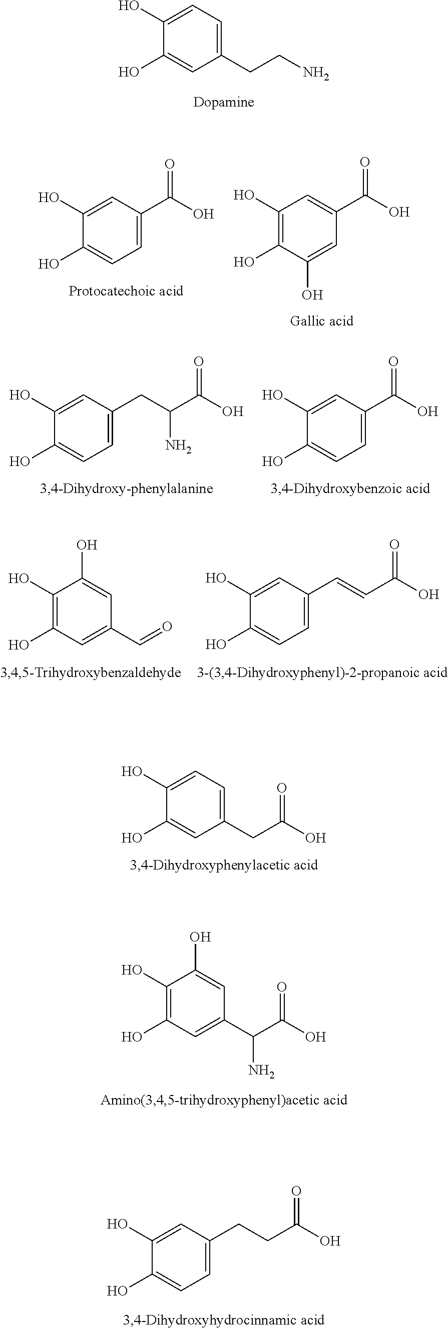

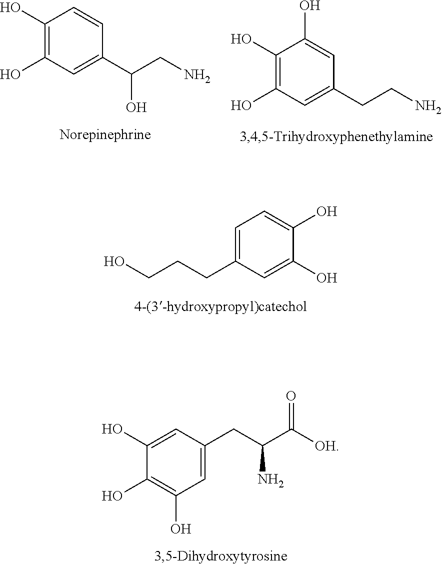 [SMILES: NCC(O)c1ccc(O)c(O)c1 ; NCCc1cc(O)c(O)c(O)c1 ; OCCCc1ccc(O)c(O)c1 ; N[C@@H](Cc1cc(O)c(O)c(O)c1)C(=O)O]

In some embodiments, the hydroxyls on these compounds may be protected to further favor the amine reaction with the isocyanate, and then deprotected to retain the adhesive function.

The diisocyanate molecule may be selected from:

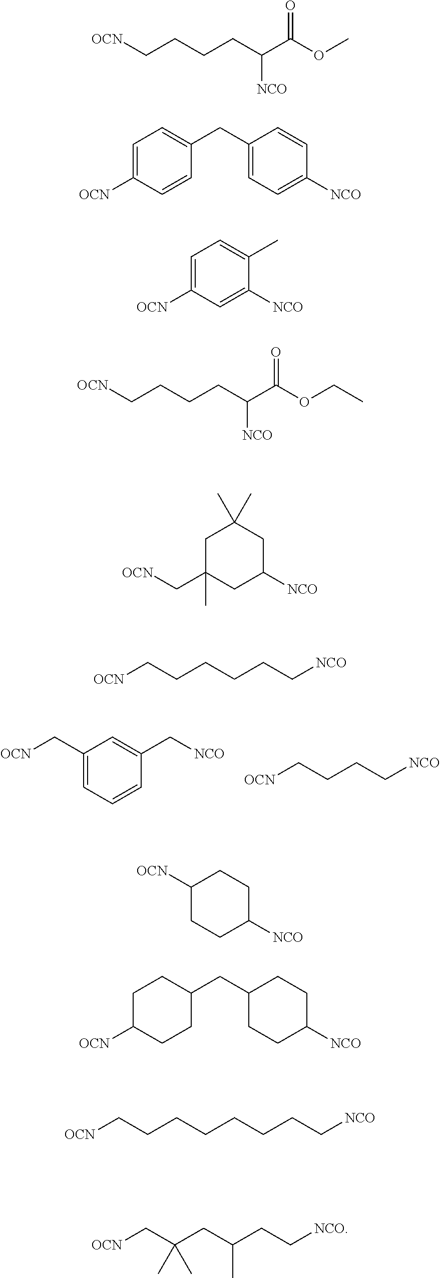

Also provided are amphiphilic oligomers prepared according to processes disclosed herein.

In some embodiments, the ratio of precursor of the hydrophobic segment to precursor of the hydrophilic segment is between 0.3 and 2.1, preferably between 0.3 and 1.0.

Also provided are methods of formulating therapeutic agents using amphiphilic oligomers disclosed herein. One such method comprises: mixing a therapeutic agent with an aqueous solution comprising adhesive amphiphilic oligomers as disclosed herein and a hydrogen peroxide scavenger or antioxidant; and allowing the amphiphilic oligomers to assemble into a hydrogel encapsulating the therapeutic agent. These methods can further include delivering the hydrogel encapsulating the therapeutic agent and the hydrogen peroxide scavenger or antioxidant to a target location within a patient body, wherein the adhesive groups of the amphiphilic oligomers cross-link at the target location. The therapeutic agent may be a small molecule drug, a protein or protein derived peptide, a nucleotide or an oligo nucleotide.

In another aspect, there is provided a composition comprising amphiphilic oligomers having terminal adhesive groups, preferably selected from terminal benzene-1,2-diol derivatives and/or terminal adhesive benzene-1,2,3-triol derivatives, and a catalase, wherein the amphiphilic oligomers are capable of self-assembling into a gel in aqueous solution and wherein cross-linking of the terminal adhesive groups releases hydrogen peroxide.

Many further features and combinations thereof concerning the present improvements will appear to those skilled in the art following a reading of the instant disclosure.

### DESCRIPTION OF THE DRAWINGS

FIG. 1 is a brief overview of a 3-step synthesis to obtain amphiphilic adhesive oligomers (AAOs), where A and B are the polyols (PEG and PCL respectively), C is the LDI, and D is denoted as an amine-terminated functional group represented as (D-C-A-C)ₙ—B, where n=2 or 3 is dependent on the number of hydroxyl groups on the polyol backbone. In Table 1, AAO_3 and AAO_7 followed an opposite sequential addition, where PCL was denoted as A and B was PEG.

FIG. 2 is a schematic highlighting the fractional isocyanate conversion with respect to time, in hours, to synthesize AAO_1. The results reflect the four repeats of the reaction. The first reaction is shown with circles, the second with squares, the third with triangles, and the last with diamonds. In addition, the two black lines represent the targeted isocyanate consumption at 50 and 75%. Error bars represent the standard deviation where three replicates of the titration were performed for each time point (N=4, n=3).

FIG. 3 is a schematic highlighting the fractional isocyanate with respect to time, in hours, to synthesize AAO_2. The results reflect the four repeats of the reaction. The first reaction is shown with circles, the second with squares, the third with triangles, and the final repeat with diamonds. In addition, the two black lines represent the targeted isocyanate consumption at 50 and 75%. Error bars represent the standard deviation where three replicates of the titration were performed for each time point (N=4, n=3).

FIG. 4 is a schematic highlighting the fractional isocyanate conversion with respect to time, in hours, to synthesize AAO_3. The results from the first reaction is shown with circles (where PEG was added at 1 hr), and the second with squares (where PEG was added at 0.5 hr). In addition, the two black lines represent the targeted isocyanate consumption at 50 and 75%. Error bars represent the standard deviation where three replicates of the titration were performed for each time point (N=2, n=3).

FIG. 5 is a schematic highlighting the fractional isocyanate conversion with respect to time, in hours, to synthesize AAO_4. Only one synthesis is shown in black circles. The two black lines represent the targeted isocyanate consumption at 50 and 75%. Error bars represent the standard deviation where three replicates of the titration were performed for each time point (N=1, n=3).

FIG. 6 is a schematic highlighting the fractional isocyanate conversion with respect to time, in hours, to synthesize AAO_7. Only one synthesis was shown in circles. The two black lines represent the targeted isocyanate consumption at 50 and 75%. Error bars represent the standard deviation where three replicates of the titration were performed for each time point (N=1, n=3).

FIG. 7 is a schematic highlighting the fractional isocyanate conversion with respect to time, in hours, to synthesize AAO_8. Two independent syntheses are shown in circles and squares, where the two black lines represent the targeted isocyanate consumption at 50 and 75%. Error bars represent the standard deviation where three replicates were of the titration performed for each time point (N=2, n=3).

FIG. 8 is a schematic highlighting the fractional isocyanate with respect to time, in hours, to synthesize AAO_9. One synthesis is shown (black circles). The two black lines represent the targeted isocyanate consumption at 50 and 75%. Error bars represent the standard deviation where three replicates of the titration were performed for each time point (N=1, n=3).

FIG. 9 is the concentration of catechol species detected quantitatively by UV-Vis at 280 nm with respect to the number of washes during the purification process. Error bars represent the standard deviation where three replicates were completed for AAO_1 performed for each wash (N=1, n=3), whereas there was no replication completed for AAO_2 (N=1, n=1).

FIG. 10 is a photo that depicts the concentration of catechol species detected during each purification process in a qualitative methodology with the addition of NaOH into the aqueous phase. (A) Calibration samples of dopamine hydrochloride between 0.05 to 0.001 wt. % dissolved in Milli-Q water with the addition of 10 N NaOH. After the addition of NaOH to each of the aqueous washes, the concentration of catechols were labelled according to the calibration curve (B) of AAO_1 and (C) AAO_2. Catechol concentration was the highest and lowest at the 1^{st} and last wash.

FIG. 11 is ¹H-NMR spectra in d-DMSO of the aqueous phase after the first wash of (A) AAO_1 and (B) AAO_2 with the corresponding residual reagent and by-products (C) the structure of activated dopamine and (D) DOPA-LDI-DOPA, which are both water-soluble. Additionally, overlapping peaks associated with the AAO_1 and AAO_2 derived water washes were observed with triethylamine hydrochloride (TEA HCl at 1.18 ppm and 3.11 ppm) and residual water (H₂O at 3.3 ppm) in the two samples.

FIG. 12 is the (A)¹H-NMR result of the purified AAO_1 labelled with unique peaks present on the spectra. All integrations were normalized to peak 4 on the catechol at 3.13 ppm (NH—OC—NH—CH₂—CH₂—). Notable peaks are peak 5 on PEG at 3.51 ppm (O—CH₂—CH₂—O), peak 2 on PCL at 2.29 ppm (O—CH₂—CH₂—CH₂—CH₂—CH₂—COO), and lastly, peak 6 on LDI at 3.6 ppm (CH₃—O—CO—CH). (B) Identifies the chemical groups that correspond to each peak from (A) for the monomer in the oligomer structure. The protons from Table 2 show the actual protons from the spectra and the theoretical value according to the ideal synthesis conditions.

FIG. 13 is the (A)¹H-NMR result of the purified AAO_2 labelled with unique peaks present on the spectra. All integrations were normalized to peak 4 on the catechol at 3.13 ppm (NH—OC—NH—CH₂—CH₂—). Notable peaks are peak 5 on PEG at 3.51 ppm (O—CH₂—CH₂—O), peak 2 on PCL at 2.29 ppm (O—CH₂—CH₂—CH₂—CH₂—CH₂—COO), and lastly, peak 6 on LDI at 3.6 ppm (CH₃—O—CO—CH). (B) Corresponds each peak from (A) to the monomer in the oligomer structure. The protons from Table 2 showed the actual protons from the spectra and the theoretical value according to the ideal synthesis conditions.

FIG. 14A shows the numbers from the homonuclear correlation spectroscopy (COSY) corresponding to the spectra from FIG. 12 and FIG. 13 of AAO_1 (FIG. 14B) and AAO_2 (FIG. 14C), respectively, where the two spectra are very similar (except for peak 1 on AAO_2). The solid black and dotted black lines correspond to the protons on dopamine and LDI, respectively. Peak 9 at 2.4 ppm (—NH—OC—NH—CH₂—CH₂—) correlates with peak 4 at 3.1 (—NH—OC—NH—CH₂—CH₂—), which correlates with the secondary amine on urea from peak 10 at 5.6 ppm (—NH—OC—NH—CH₂—CH₂—). The other secondary amine from the urea is found at peak 11 at 5.8 ppm (—CH₂—NH—OC—NH—), which correlates with peak 3 (and 4) from the aliphatic group on LDI at 2.9 ppm (—CH₂—CH₂—NH—OC—NH—). On the opposite end of the LDI, urethane peaks are denoted as peak 19 and 21 and correlate with the tertiary carbon on LDI at peak 18 at 3.9 ppm (—NH—CH₂—CH₂—CH₂—CH₂—CH—NH—CO—) and the secondary carbon on LDI at peak 3 at 2.9 ppm (—OC—NH—CH—CH₂—CH₂—CH₂—CH₂—NH—OC—NH—), respectively.

FIG. 15 is a synthesis schematic of AAO_1 and AAO_2.

FIG. 16 is a photo that depicts the remaining oligomers that were not further characterized due to incomplete solubilization following overnight stirring on ice. Labelled from left to right are (A) AAO_3 (B) AAO_4 (C) AAO_7 (D) AAO_8 and (E) AAO_9.

FIG. 17 is a photo that depicts AAO_2 injected into water at 4° C. (A: left) and 37° C. outlined in the black circle (B: right), respectively.

FIG. 18 is a photo that depicts a 4° C. solution of (A) AAO_2, (B) AAO_5 and, (C) AAO_6 that was loaded into a 1 mL syringe with an 18-gauge needle and injected into 37° C. PBS, resulting in (A) a phase-separated gel circled in black, while (B) and (C) resulted in a translucent gel is labelled in a black box. These photos illustrated how the terminal domain of DOPA, MeOH, or F-DOPA contributes to the gelling mechanism of the oligomer.

FIG. 19 is the oxidation process of catechol in PBS that forms intermediate products and can be captured by the UV-Vis

FIG. 20 is the absorbance measurements recorded on the UV-Vis of (A) AAO_1 at 0.0075 wt. % and (B) AAO_2 at 0.05 wt. % with respect to the black time curve. Also, the intermediate products correspond to their wavelength, illustrated with a black arrow.

FIG. 21 is the differential scanning calorimetry (DSC) thermogram of AAO_1 (n=1). Heating and the cooling rate at 10° C./min from −90 to 160° C. The analysis was completed on NEXTA Standard Analysis software (Ver 2.0.0.5). The numbering on the curve corresponds to the sequence of the cycle starting with 1.

FIG. 22 is the DSC thermogram of AAO_2 (n=1). Heating and the cooling rate at 10° C./min from −90 to 160° C. The analysis was completed on NEXTA Standard Analysis software (Ver 2.0.0.5). The numbering on the curve corresponds to the sequence of the cycle starting with 1.

FIG. 23 is the DSC thermogram of AAO_6 (n=1). Heating and the cooling rate at 10° C./min from −90 to 160° C. The analysis was completed on NEXTA Standard Analysis software (Ver 2.0.0.5). The numbering on the curve corresponds to the sequence of the cycle starting with 1.

FIG. 24 is the DSC thermogram of AAO_1 (n=1) characterized to validate the melt peaks were not associated with solvents. The heating and cooling rate was at 10° C./min from −90 to 160° C. The analysis was completed on NEXTA Standard Analysis software (Ver 2.0.0.5). The numbering on the curve corresponds to the sequence of the cycle starting with 1.

FIG. 25 is the swelling/degradation profile of AAO_1 (black circles) and AAO_2 (black squares) at 20 wt. % in simulated synovial fluid (n=2). Data are reported with standard deviations.

FIG. 26 is a proposed physical and chemical crosslinking mechanism of AAO_1 and AAO_2. PCL, PEG-LDI and DOPA are represented by dashed lines, tilde, and circle, respectively. The solid black lines suggest the chemical crosslinking between randomized catechol domains.

FIG. 27 is a typical strain (%) versus stress (Pa) graph for AAO_1 (in the solid black line) and AAO_2 (in the dotted black line) (after three days of curing in PBS in a 37° C. oven) of a gel to illustrate changes in the compressive moduli (n=1).

FIG. 28 is a schematic of the biocompatibility experiment for AAO_1 and AAO_2 with three conditions; (1) media, (2) catalase, and (3) catalase. On Day 1, condition 3 was changed to media without catalase, while the remaining two conditions used media or media supplemented with 100 U/mL of catalase. Water-soluble tetrazolium salt (WST) assessed leachable content from days 1, 3, and 7.

FIG. 29 is a graph summarizing the concentration of hydrogen peroxide by the FOX Assay quantified by the Pierce™ Quantitative Peroxide Assay Kit by Thermo Scientific. The calibration curve of hydrogen peroxide was diluted with complete cell culture media between 1-100 μM. The absorbance was measured on a Plate Reader at 570 nm. The equation relating the concentration of hydrogen peroxide to absorbance was y=0.0141x+0.0598 with an R² of 0.981.

FIG. 30 is the metabolic activity for primary chondrocytes exposed to extracts from the gels at a ratio of 1:80 (gel:media) for 24 hours. Each data set has been normalized to the negative control of the growth media of 100%. Error bars represent the standard deviation calculated from three replicates performed for each condition (N=1, n=3), where p<0.0001 is signified by **** compared to media.

FIG. 31 is the metabolic activity of primary human chondrocytes cultured in extracts from the adhesives. AAO_2UP was incubated with cell culture media at a ratio of 1:80 for 24 hours. Each data set has been normalized to the negative control of the growth media. Error bars represent the standard deviation calculated from three replicates performed for each condition (N=1, n=3).

FIG. 32 is the metabolic activity of chondrocytes exposed with H₂O₂ between 25 and 100 μM. The data has been normalized to the negative control of chondrocytes cultured with media alone at 100%. The error bars correspond to the standard deviations (N=1, n=3), and *** is signified by p<0.001 with reference to the negative control of media.

FIG. 33 is the metabolic activity for primary chondrocytes exposed to four conditions: the negative control of media, positive control of 1% Triton, AAO_1, and AAO_2. Each condition was supplemented with 200 μM ascorbic acid, 100 U/mL catalase, or no modification to the media. The results from the three conditions have been normalized to the corresponding media control with chondrocytes. The error bars correspond to the standard deviations (N=1, n=3), and ***** is signified by p<0.0001 with reference to the catalase condition for the corresponding gels.

FIG. 34 is the metabolic activity for primary chondrocytes presented as a percentage (N=3) for AAO_1 (A) and AAO_2 (B) for each condition (C1, C2, and C3) at each day point (D1, D3, and D7). All the results were normalized to cells without treatment with minimum metabolic activity specified by the dashed black line. Three phase-separated gels were performed at each condition and day, additionally, the experiment was replicated three times. Errors bars represent standard deviations (N=3, n=3). * and **** represents p<0.05 and p<0.0001, respectively with reference to Day 1 for the corresponding conditions, meanwhile && is p<0.01 with reference to Day 7 C2.

FIG. 35 is the calibration curve of celecoxib solubilized in 50:50 Methanol/SSF between 10.0 and 0.001 μg/mL analyzed via the ultra performance high liquid chromatography.

FIG. 36 is the in vitro release of roughly 5.5 wt. % of celecoxib (with respect to the oligomer) encapsulated into 20 wt. % of AAO_1 (o) and AAO_2 (□). The release was conducted for 42 days in SSF with 0.02 wt. % Tween™ 80. Error bars represent the standard deviation calculated from three separate gels corresponding to each oligomer (N=3, n=1).

FIG. 37 is the production of hydrogen peroxide of AAO_1 (in circle) and AAO_2 (in square) (N=1) quantified by Pierce™ Quantitative Peroxide Assay Kit with respect to days by Thermo Scientific. Error bars represent the standard deviation where three replicates were performed for each time point (N=1, n=3).

### DETAILED DESCRIPTION

Provided herein are amphiphilic oligomers with thermoresponsive and adhesive properties. Following the formation of a gel, evidence of chemical crosslinking was observed in the absence of a secondary crosslink activator normally used for such systems.

Hydrogels are a hydrophilic three-dimensional polymeric network with known capabilities to retain large amounts of water. Due to their inherent composition, hydrogels can resemble biological tissues' physical and mechanical properties, including the cartilage or meniscus. Hydrogels are three-dimensional matrices mainly comprised of water, while the remainder is polymeric-based. These vehicles are excellent candidates for drug delivery systems as they can readily load and deliver therapeutic agents or growth factors. Other biomedical applications include entrapment of cells to promote differentiation since hydrogels can mimic the 3-D ECM of biological tissues due to their similar compositions

Per the Examples, novel oligomers were synthesized to yield phase-separated gels formed with an amphiphilic backbone derived from PEG and a degradable hydrophobic domain consisting of PCL. These were linked with an isocyanate group, while the terminal domains on the oligomer were functionalized with dopamine.

Chemically crosslinked hydrogels are desirable due to their elevated mechanical properties, though this crosslinking can be a shortcoming as it can immediately plug the polymer within an injection needle. Alternatively, if the gelation is too slow, this would prevent the formation of the 3D matrix as the precursor and/or encapsulated molecules would diffuse into the injected surroundings. Importantly, unreacted functional groups, crosslinkers, catalysts, or initiators can lead to toxicity concerns.

Physically crosslinked hydrogels have been extensively studied. Traditionally, these form by a change in the surroundings, such as temperature or pH. Of particular interest for biomedical applications are thermoresponsive polymers, in part because they can directly and immediately change from liquid to a solid as the temperature transitions from the ambient environment to that of 37° C. Thermoresponsive polymers are a liquid below their lower critical saturated temperature (LCST) and can form a solid viscoelastic hydrogel above this temperature. Clinically, this gelation mechanism is useful since the hydrogel can form within the body circumventing invasive procedures to implant a gel.

As will be apparent with reference to the Examples, the gels taught herein may have some of the advantages of both chemically and physically crosslinked hydrogels known in the art.

As used herein, an oligomer “derived” from an identified polymer retains the essential structure and activity of the polymer despite any modifications thereto. In various embodiments, the oligomer chain is selected from the identified biocompatible polymers.

In some embodiments, the amphiphilic oligomer has a molecular weight of between 4400 and 10,000, in some embodiments, between 5000 and 8000.

The amphiphilic oligomers include at least one hydrophobic segment derived from a degradable hydrophobic polyol having a molecular weight between 900 and 2000, preferably between 800 and 1400. In some embodiments, the polyol is a PCL diol or triol.

In various embodiments, the hydrophobic segment is a degradable hydrophobic segment derived from a polyester (e.g. polycaprolactone, polylactic acid, polyglycolic acid, polycaprolactone), a polycarbonate, polyamide, polyurethane (PU), cellulosic oligomer, oligosaccharide, poly(alkenedicarboxylate) (e.g. poly(butylene succinate), poly(hydroxybutyrate), poly anhydrides, poly peptides, poly(p-hydroxyalcanoate), poly(hydroxybutyrate-co-hydroxyvalerate) or Poly(p-dioxanone).

The amphiphilic oligomers include at least one hydrophilic segment derived from a hydrophilic polyol having a molecular weight between 600 and 2000, preferably between 800 and 1200.

In various embodiments, the hydrophilic segment is derived from polyvinyl alcohol (PVA), polyvinylpyrrolidone (PVP), polyethylene oxide (PEO), co-poly(ethylene oxide)-b-poly(propylene oxide) poly(hydroxyethylmethacrylate) (polyHEMA), or poly acrylic acid or poly acrylic acid mono-salts (H⁺ substituted for Na⁺, K⁺).

In one embodiment, the isocyanate component is a linear diisocyanate.

In one embodiment, the diisocyanate is derived from lysine. In one embodiment, the diisocyanate is lysine diisocyanate (LDI).

In various embodiment, the diisocyanate molecule may be selected from the group consisting of:

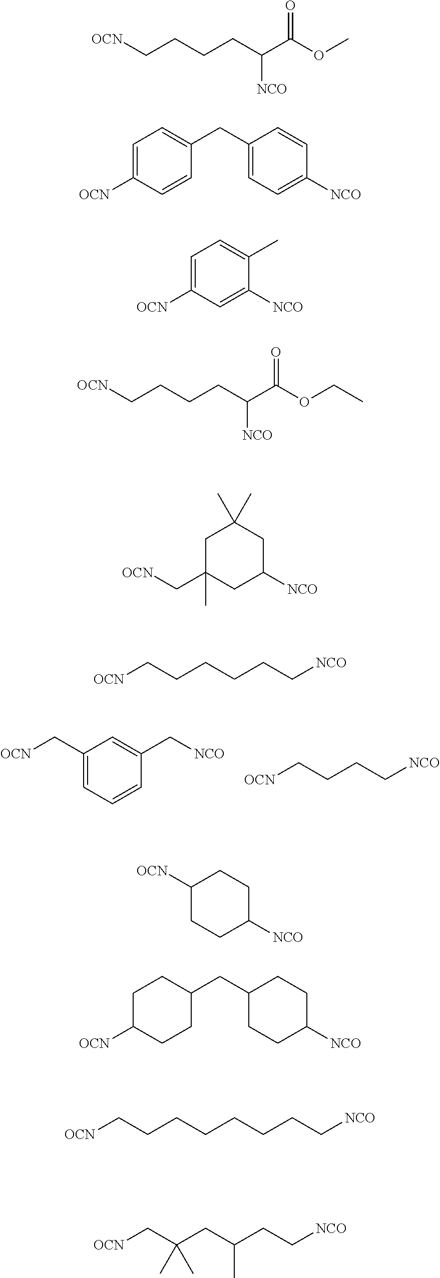

In preferred embodiments, the diisocyanate is one of:

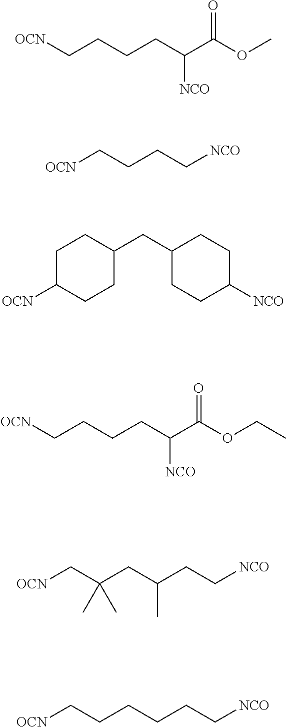

A novel synthesis strategy is provided to form amphiphilic oligomers linked with isocyanates and terminal adhesive (catechol) groups.

The terminal groups may be a 4-alkylbenzene-1,2-diol derivative or a 5-alkylbenzene-1,2,3-triol derivative selected from the group consisting of:

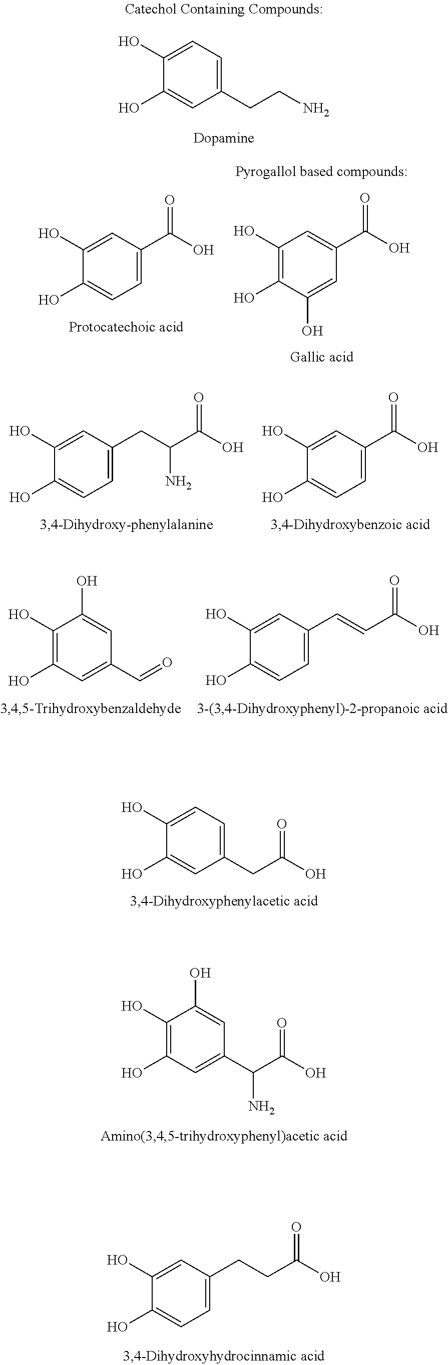

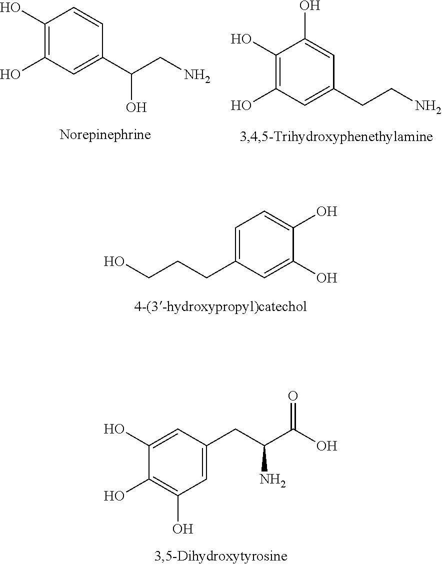 [SMILES: NCC(O)c1ccc(O)c(O)c1 ; NCCc1cc(O)c(O)c(O)c1 ; OCCCc1ccc(O)c(O)c1 ; N[C@@H](Cc1cc(O)c(O)c(O)c1)C(=O)O]

(Referenced herein generally as catechols.) In some embodiments, the hydroxyls on the catechols may be protected to further favor the amine reaction with the isocyanate, and then deprotected to retain the catechol adhesive function. Suitable protecting groups are known to those of skill in the field.

Traditionally, catechol hydrogels are formed due to the addition of a chelating agent or an oxidant to initiate the chemical crosslinking mechanism. The degree of crosslinking depends heavily on the concentration of the crosslinker and the number of catechol species.

In the absence of oxidants and chelating agents, catechols oxidize via an autoxidation mechanism to form o-quinone, which can further react with amines and thiols found on tissue via the Michael-Type addition or a Schiff base reaction [see J. Yang, M. A. Cohen Stuart, and M. Kamperman, Jack of all trades: versatile catechol crosslinking mechanisms, Chem Soc Rev, vol. 43, no. 24, pp. 8271-98, Dec. 21 2014; M. J. LaVoie, B. L. Ostaszewski, A. Weihofen, M. G. Schlossmacher, and D. J. Selkoe, “Dopamine covalently modifies and functionally inactivates parkin,” Nat Med, vol. 11, no. 11, pp. 1214-21, November 2005; J. H. Ryu, Y. Lee, W. H. Kong, T. G. Kim, T. G. Park, and H. Lee, “Catechol-functionalized chitosan/pluronic hydrogels for tissue adhesives and hemostatic materials,” Biomacromolecules, vol. 12, no. 7, pp. 2653-9, Jul. 11 2011]. Additionally, catechols offer cohesive properties due to the oxidization process forming an inherent chemical crosslinked matrix [Yang, M. A. Cohen Stuart, and M. Kamperman, “Jack of all trades: versatile catechol crosslinking mechanisms,” Chem Soc Rev, vol. 43, no. 24, pp. 8271-98, Dec. 21 2014]. The chemical crosslinking is beneficial to delay bulk-erosion of the gel and offer a sustained release of a therapeutic agent, unlike a physically crosslinked gel.

Following the formation of a gel, evidence of chemical crosslinking was observed in the absence of a secondary crosslink activator normally used for such systems. Per the Examples, the phase-separated gel may include a number of desirable features, including: (1) similar compressive modulus value to a human tissue (e.g. meniscus), (2) a relatively constant drug release profile between 5 and 42 days, and (3) a stable chemical crosslinked system without a change in the wet mass, in one embodiment for at least 21 days.

For the purposes of gel formation, the ratio of hydrophobic to hydrophilic content in the oligomer should fall within a certain ratio. If the hydrophobic content is too high, the polymer will not be soluble in aqueous medium (the gelation medium) at low temperature and thus will not be an injectable fluid. The hydrophilic moiety enables the oligomers to dissolve at low temperatures (in preferred embodiments at a temperature of 4° C.), to generate the injectable liquid, which when reaching sufficiently high temperatures (in preferred embodiments, >20° C., will undergo a reverse state (from viscous fluid to solid gel), with the hydrophobic moieties gaining enough energy to move around and self assemble (phase separate from hydrophilic component), thereby inducing a precipitated hydrophobic aggregate. If the hydrophobic ratio is too low then the polymer will not precipitate out at elevated temperature (i.e. >20° C.) and remain soluble. The ratio of hydrophobic segment to hydrophilic segment is suitably between 0.3 and 2.1, preferably 0.3 and 1.0

In some embodiments, compositions and gels as provided herein include one or more fillers. In this regard, mechanical properties can be increased with the addition of fillers and certain suitable fillers will be known to those of skill in the art. In one embodiment, the compositions and gels as provided herein may include nano or microparticle carriers. In some embodiments, therapeutic agents are associated with, in some embodiments, encapsulated within, suitable nanoparticles. Examples of drug loaded nanoparticles are available in the literature. Patra, J. K. et al., “Nano based drug delivery systems: recent developments and future prospects”, Journal of Nanobiotechnology, 16: 71 (2018), for example, discloses a number of nanomaterials and nano based drug delivery systems that may be used.

As detailed in the Examples, cross-linking of the adhesive terminal groups can be associated with the production of hydrogen peroxide by which the toxic effects may be mitigated by including a H₂O₂ scavenger within the gel. In one embodiment, catalase is included within the gel. Importantly, the inventors determined that catalase is not denatured by the polymers exemplified herein, making it available to degrade H₂O₂ generated by the crosslink formation between the adhesive (DOPA) groups. Other such scavengers may include small molecules such as vitamin-C and vitamin-E.

Protein adsorption by polymers often results in significant conformational changes, which can affect protein interaction with ligands, substrates, and antigens, which are dependent on the orientation of the binding site of interest. These conformational changes, as a result of protein adsorption, can also denature the protein and change its native properties [see Firkowska-Boden, I. et al., “Controlling Protein Adsorption through Nanostructured Polymeric Surfaces”, Advanced Healthcare Materials. 7 (1): (2017), 1700995. doi:10.1002/adhm.201700995. PMID 219399 Studies have demonstrated both denaturation of adsorbed protein and deactivation of enzymes.

In the context of DOPA-modified polymers, most researchers have tried to conceive of methods to avoid H₂O₂ production with a secondary crosslinker to specific DOPA reactions and avoid H₂O₂ production. However, the Examples demonstrate that, surprisingly, catalase is not deactivated by the amphiphilic oligomers taught herein and, further, that the catalase does not interfere with the aggregation into gels or the subsequent adhesive group (DOPA) crosslinking. By providing a polymer system that crosslinks in the presence of catalase and demonstrating that the use of catalase enhances biocompatibility, the present inventors have provided a novel mechanism to eliminate both the use of secondary crosslinkers and the accumulation of H₂O₂.

In some embodiments, there is provided pharmaceutical compositions that include amphiphilic oligomers and an aqueous carrier, or a self-assembled hydrogel, as described herein and a therapeutic agent. The therapeutic agent (e.g. a small molecule drug) may be encapsulated within the hydrogel. As used herein, “encapsulated” refers to confining a compound or composition therewithin and can encompass both complete and partial encapsulation, but in one embodiment complete encapsulation.

In some embodiments, the gel itself may be used for therapeutic or cosmetic purposes, and in such latter applications may or may not include a pharmaceutical agent.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

As used herein “subject” refers to an animal being administered a therapeutic, in one embodiment a mammal, in one embodiment a human patient. As used herein “treatment”, and grammatical variations thereof, refers to administering a compound or composition of the present invention. The treatment may require administration of multiple doses, which may be at regular intervals

In some embodiments, a single administered dose of a sustained release pharmaceutical composition as described herein provides localized effects for a period of at least 24 hours, at least 48 hours, at least 1 week, or at least 3 weeks.

The pharmaceutical compositions as described herein are particularly advantageous for applications for direct delivery to a treatment site, which can maximize bioavailability and decrease systemic complications.

The pharmaceutical compositions described herein may be injectable, wherein injection may be, for example, by syringe, via a catheter or other device for delivering a liquid material across the skin. The composition may be administered by injection by ejecting the material from a syringe with or without a needle, (e.g. into an open wound in some embodiments.)

In some embodiment, the composition has a viscosity sufficient to be expelled out of a syringe at room temperature. In some embodiments the composition has a viscosity sufficient to be expelled out of a syringe at 10° C., in some embodiments at 4° C. In some embodiments, the composition has a viscosity sufficient to be expelled from a needle having a gauge between 7 gauge to 33 gauge or more specifically 18 gauge.

When administered via injection, the composition can operate as a depot injection, the composition forming a localized mass. In one embodiment the composition is administered by a single injection. The pharmaceutical compositions as described herein may be administered in a number of ways depending upon the area to be treated. Without limiting the generality of the foregoing, in a particular embodiment, the compositions are administered by subcutaneous, intradermal or intramuscular injection. In one embodiment, the pharmaceutical composition is administered as an intra-articular depot. The pharmaceutical composition as described herein may conveniently be presented in unit dosage form of a single-use syringe that has been sterilized for injection with or without a needle.

In one embodiment, a treatment is administered in order to provide localized pain relief. This treatment may be to alleviate pain or the use may be prophylactic to prevent pain. In one embodiment, there is provided a method of treating or preventing pain comprising administering, preferably by injection, a therapeutically effective amount of a pharmaceutical composition as described herein.

Without limiting the generality of the foregoing, the present compositions may have particular utility in association with the treatment of osteoarthritis.

All documents referenced herein are incorporated by reference, however, it should be appreciated that any patent, publication, or other disclosure material, in whole or in part, that is incorporated by reference herein is incorporated only to the extent that the incorporated material does not conflict with definitions, statements, or other disclosure material set forth in this disclosure. As such, and to the extent necessary, the disclosure as explicitly set forth herein supersedes any conflicting material incorporated herein by reference.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

### EXAMPLES

### Example 1: Synthesis of Oligomers

All chemicals were purchased from Sigma-Aldrich unless otherwise stated.

Statistical analysis was performed using GraphPad Prism Software (Version 7.00) and analyzed by analysis of variance (ANOVA) using Tukey's test or an independent sample t-test. Statistical significance was assigned for p<0.05 for all experiments.

### 1.1 General Synthesis Strategies of Oligomers

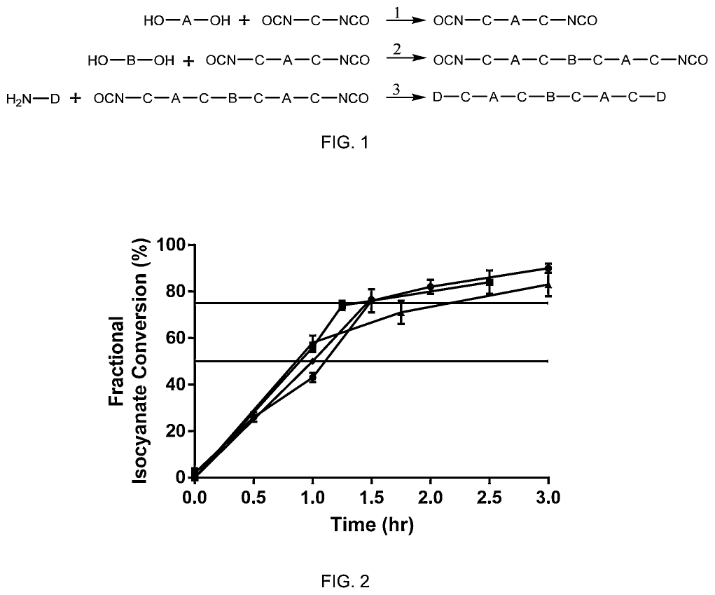

A methodical approach was developed to synthesize AAO molecules utilizing PEG and PCL, lysine diisocyanate (LDI), and end terminal groups of either dopamine hydrochloride (DOPA), 3,4-dimethoxyphenethylamine (F-Dopa), or methanol (MeOH), along with the addition of dibutyltin dilaurate (DBTDL) as a catalyst. Triethylamine (TEA, anhydrous 99.5%) was added to remove the hydrochloride salt from DOPA. N,N-dimethylacetamide (DMAc, anhydrous 99.8) was used as the solvent to solubilize all monomers. The oligomeric reagents and their corresponding molecular weights, hydroxyl functionalities of the polyol, and the nature of the end terminal groups for each AAO formulation are summarized in Table 1. A general synthesis schematic for AAO oligomers is shown in FIG. 1.

This Example provides a novel synthesis strategy for preparing AAO oligomers and a family of oligomers by adjusting the molecular weights of the polyols and changing the order of the polyol addition.

TABLE 1
A summary table of each oligomer is included for a given synthesized AAO,
along with the polyol's respective average number molecular weight, number
of alcohol functionalities, and the terminal end groups. AAO_5 and AAO_6,
which lack terminal adhesive groups are controls.

| | PEG | | | PCL | | | End Terminal Group | | |
|---|---|---|---|---|---|---|---|---|---|
| | 600 - | 1000 - | 2000 - | 1250 - | 900 - | 2000 - | | Fake | |
| Legend | Diol | Diol | Diol | Diol | Triol | Triol | DOPA | DOPA | MeOH |
| AAO_1 | | X | | X | | | X | | |
| AAO_2 | | X | | | X | | X | | |
| AAO_3 | | X | | X | | | X | | |
| AAO_4 | | | X | X | | | X | | |
| AAO_5 | | X | | | X | | | X | |
| AAO_6 | | X | | | X | | | | X |
| AAO_7 | | | X | X | | | X | | |
| AAO_8 | | X | | | | X | | | |
| AAO_9 | X | | | | X | | X | | |

Initially, the polyols were degassed 24 hours before the reaction, under reduced pressure at 0.1 mm Hg, while stirring on a hot plate (60° C.). LDI was distilled at 120° C. at reduced pressure, and the final product was stored under nitrogen at −20° C. On the day of the reaction, PEG and PCL were diluted with DMAc (molarity values are reported in the respective synthesis description) outside of the glovebox. Dibutylamine (DBA) and trichlorobenzene (TCB) were used to determine the kinetics of the isocyanate conversion [S. Sharifpoor, R. S. Labow, and J. P. Santerre, “Synthesis and characterization of degradable polar hydrophobic ionic polyurethane scaffolds for vascular tissue engineering applications,” Biomacromolecules, vol. 10, no. 10, pp. 2729-39, Oct. 12 2009; H. Staley and D. J. David, Analytical chemistry of the polyurethanes. New York, Robert E. Krieger, 1979]. The glovebox was purged eight times with nitrogen to minimize the exposure to moisture from the air. To begin the reaction, LDI, DMAc, and DBTDL (0.4 mol % relative to the isocyanate groups) were added into a round bottom flask and stirred on a hot plate at 40° C. to yield a 0.69 M concentration of LDI. The first polyol was added dropwise from a syringe between a 6-to-9-minute period into the LDI solution. Once 50% conversion was obtained, validated by the isocyanate titration, the second polyol was added dropwise to achieve 75% conversion shown in Step 2 of FIG. 1. The terminal groups of isocyanates were quenched with dopamine hydrochloride, methanol, or F-DOPA. Methanol and F-Dopa were diluted with DMAc and added directly into the oligomer. Dopamine hydrochloride was first pre-activated with triethylamine to remove the hydrochloride salt [L. Q. Xu, H. Jiang, K.-G. Neoh, E.-T. Kang, and G. D. Fu, “Poly (dopamine acrylamide)-co-poly (propargyl acrylamide)-modified titanium surfaces for ‘click’functionalization,” Polymer Chemistry, vol. 3, no. 4, pp. 920-927, 2012]. Dopamine hydrochloride was dissolved in DMAc for 5 minutes to yield a 0.5 M solution. Next, triethylamine (TEA) at a 0.95:1 stoichiometric molar ratio with dopamine hydrochloride was stirred for 15 minutes, in which the solution transitioned from a transparent to opaque colour since triethylamine hydrochloride precipitated from DMAc. The solution was centrifuged at 3420 rpm for 30 minutes to ensure the triethylamine hydrochloride was further precipitated by forming a pellet while collecting the supernatant. Before the last addition, the round bottom flask was removed from the glovebox and cooled to room temperature by placing it on ice. The DMAc solution with the terminal functional groups (DOPA, F-Dopa, or MeOH) was gradually added into the oligomer and stirred overnight on ice to ensure all isocyanate groups had reacted.

Upon completing the reaction, the final product was filtered using a Buchner Funnel to remove the remaining triethylamine hydrochloride before precipitation in ethyl ether. The oligomer was added dropwise into 2 L of ethyl ether (Anhydrous, Caledon) spinning at 800 rpm at room temperature, where the oligomer was precipitated from DMAc, and the catalyst was extracted from the oligomer. The solution was stirred overnight at room temperature until the diethyl ether solution turned transparent to ensure the oligomer had completely precipitated. The supernatant was decanted, and approximately 10 mL of chloroform (Caledon) was added to dissolve the product. The product was then reprecipitated in 1 L of ethyl ether. This process was repeated three times to minimize the presence of the tin catalyst and DMAc. To remove residual triethylamine hydrochloride, free dopamine, and other potential dimers or trimers, water washes were conducted at room temperature. The product was dissolved in approximately 50 mL of chloroform, where 5 mL was added into a 50 mL falcon tube. Next, 40 mL of Milli-Q water was added to the falcon tube, where the solution was vortexed for a minute and then centrifuged at 3420 rpm at −20° C. The oligomer was more readily soluble in chloroform than water which effectively removed all water-soluble monomers during this liquid-liquid extraction. On the last wash, ethyl ether was added to straight-sided amber glass jars (Kimble Chase, VWR), where the remaining chloroform was added dropwise for a fourth precipitation. The solvent was decanted and the precipitate was placed in a vacuum oven (Fisher Scientific Isotemp Vacuum Oven) for three days (or longer) to ensure the complete removal of all solvents.

### 1.2 Kinetics of Oligomers

The kinetics of the isocyanate reaction was monitored following a procedure from Sharifpoor and colleagues [S. Sharifpoor, R. S. Labow, and J. P. Santerre, “Synthesis and characterization of degradable polar hydrophobic ionic polyurethane scaffolds for vascular tissue engineering applications,” Biomacromolecules, vol. 10, no. 10, pp. 2729-39, Oct. 12 2009]. In short, the residual isocyanates were reacted with excess secondary amines using DBA, in which the remaining amines were back-titrated with 1.0 M hydrochloric acid (HCl) (BioShop). A 1.3 M solution of DBA in TCB was prepared in the glovebox, where 1 mL was added to individual titration vials and 5 mL of DMAc and 1 mL of the reaction mixture in which urea functionalities were formed via the reaction between the amines and the residual isocyanates. After a minimum of an hour (to ensure that all residual isocyanates have reacted with the secondary amines), 5 mL of MeOH and three drops of bromophenol blue were added to each vial. Initial and final volumes of 1.0 M HCl were recorded using the following Equation 1:

$$Fractional NCO Conversion ( {p}_{i} ) = 1 - \frac{C * [ ( {V}_{o} * \frac{{W}_{A}}{{W}_{A 0}} ) - V ]}{2 \frac{{W}_{s} {W}_{DI}}{{W}_{T} {M}_{DI}}} \qquad ( 1 )$$

Where C is the molar equivalent of HCl titrant (N), Vₒ is the volume of HCl solution used to titrate the DBA/DMAc solution (control), W_{A} is the mass of DBA solution weighed in the titration vial, W_{A0} is the mass of DBA solution weighed in the control of the titration vial, V is the volume of HCl solution used in the samples for the reaction, W_{DI} is the mass of diisocyanate added into the reaction, W_{S} is the mass of the solution from the reaction vessel added to the vial, W_{T} is the total weight in grams of solvent, polyol, isocyanate, and catalyst added into the reaction, and M_{DI} is the molecular weight of the diisocyanate.

### 1.3 Synthesis of Individual Oligomers

### AAO_1

Following Table 1, the stoichiometric ratio of reagents used to synthesize AAO_1 was LDI:PEG:PCL:DOPA in a molar ratio of 4.1:2.05:1:2.05. Briefly, PEG 1000 at a final concentration of 0.36 M in DMAc was added dropwise to LDI. Once the isocyanate conversion achieved 50%, a dropwise addition of the 0.34 M concentration of PCL 1250 (Polysciences, Inc.) was added over approximately 4 to 6 minutes until 75% was reached. The final addition of 0.45 M of activated dopamine was added simultaneously.

### AAO_2

Following Table 1, the stoichiometric ratio of reagents used to synthesize AAO_2 was LDI:PEG:PCL:DOPA in a molar ratio of 6.1:3.05:1:3.05. Briefly, PEG 1000 at a final concentration of 0.50 M in DMAc was added dropwise to LDI. Once the isocyanate conversion achieved 50%, a dropwise addition of the 0.34 M concentration of PCL 900 was added over approximately 4 to 6 minutes until 75% was reached. The final addition of 0.45 M of activated dopamine was added simultaneously.

### AAO_3

Following Table 1, the stoichiometric ratio of reagents used to synthesize AAO_3 was LDI:PCL:PEG:DOPA in a molar ratio of 4.1:2.05:1:2.05. Briefly, PCL 1250 at a final concentration of 0.29 M in DMAc was added dropwise to LDI. Once the isocyanate conversion achieved 50%, a dropwise addition of PEG 1000 at a 0.21 M concentration was added over approximately 4 to 6 minutes until 75% was reached. The final addition of 0.45 M of activated dopamine was added simultaneously.

### AAO_4

Following Table 1, the stoichiometric ratio of reagents used to synthesize AAO_4 was LDI:PEG:PCL:DOPA in a molar ratio of 4.1:2.05:1:2.05. Briefly, PEG 2000 (Polysciences, Inc) at a final concentration of 0.36 M in DMAc was added dropwise to LDI. Once the isocyanate conversion achieved 50%, a dropwise addition of PCL 1250 at a 0.27 M concentration was added over approximately 4 to 6 minutes until 75% was reached. The final addition of 0.45 M of activated dopamine was added simultaneously.

### AAO_5

Following Table 1, the stoichiometric ratio of reagents used to synthesize AAO_5 was LDI:PEG:PCL:F-Dopa in a molar ratio of 6.1:3.05:1:3.05. Briefly, PEG 1000 at a final concentration of 0.50 M in DMAc was added dropwise to LDI. Once the isocyanate conversion achieved 50%, a dropwise addition of the 0.34 M concentration of PCL 900 was added over approximately 4 to 6 minutes until 75% was reached. The final addition was F-Dopa (Acros Organics) at a concentration of 0.83 M in DMAc.

### AAO_6

Following Table 1, the stoichiometric ratio of reagents used to synthesize AAO_6 was LDI:PEG:PCL:MeOH in a molar ratio of 6:3.05:1:3.05. Briefly, PEG 1000 at a final concentration of 0.50 M in DMAc was added dropwise to LDI. Once the isocyanate conversion achieved 50%, a dropwise addition of the 0.34 M concentration of PCL 900 was added over approximately 4 to 6 minutes until 75% was reached. The final addition was MeOH at a concentration of 0.83 M in DMAc.

### AAO_7

Following Table 1, the stoichiometric ratio of reagents used to synthesize AAO_7 was LDI:PCL:PEG:DOPA in a molar ratio of 4:2.05:1:2.05. Briefly, PCL 1250 at a final concentration of 0.64 M in DMAc was added dropwise to LDI. Once the isocyanate conversion achieved 50%, a dropwise addition of PEG 2000 at a 0.25 M concentration was added over approximately 4 to 6 minutes until 75% was reached. The final addition of 0.45 M of activated dopamine was added simultaneously.

### AAO_8

Following Table 1, the stoichiometric ratio of reagents used to synthesize AAO_8 was LDI:PEG:PCL:DOPA in a molar ratio of 6:3.05:1:3.05. Briefly, PEG 1000 at a final concentration of 0.50 M in DMAc was added dropwise to LDI. Once the isocyanate conversion achieved 50%, a dropwise addition of PCL 2000 at a 0.33 M concentration was added over approximately 4 to 6 minutes until 75% was reached. The final addition of 0.45 M of activated dopamine was added simultaneously.

### AAO_9

Following Table 1, the stoichiometric ratio of reagents used to synthesize AAO_9 was LDI:PEG:PCL:DOPA in a molar ratio of 6:3.05:1:3.05. Briefly, PEG 600 at a final concentration of 0.50 M in DMAc was added dropwise to LDI. Once the isocyanate conversion achieved 50%, a dropwise addition of PCL 900 at a 0.33 M concentration was added over approximately 4 to 6 minutes until 75% was reached. The final addition of 0.45 M of activated dopamine was added simultaneously.

### Discussion

When conceiving the synthesis strategy for the AAOs, the ratio between the hydrophilic to hydrophobic moieties dictated the solubility along with the thermoresponsive properties. Thus, oligomerization between the polyols and isocyanates was minimized by adding 0.4 mol % of dibutyltin dilaurate (DBTDL) to the reaction to favour the urethane formation over side reactions with water. Additionally, other strategies to avoid oligomerization included reducing the reaction time from 24 to 0.5 or 1 hour [K. Frisch and L. Rumao, “Catalysis in isocyanate reactions,” Polymer Reviews, vol. 5, no. 1, pp. 103-149, 1970], decreasing the reaction temperature to 40° C., and lastly, the polyol was added dropwise to the LDI and DBTDL. To characterize the synthesis kinetics, isocyanate titrations were performed before and after the addition of the second polyol. This also verified that the isocyanate conversion of 50 and 75% were achieved for reaction steps 1 and 2, respectively, in FIG. 1.

### 1.4 Kinetics of AAO

### AAO_1

The kinetics observed in FIG. 2 indicated that 50% conversion of LDI functionalities with PEG occurred after one hour of the reaction, at which point PCL was added, where 75% conversion was observed between 1.25 to 1.5 hours. Values above the desired fractional isocyanate conversion suggested that residual water possibly was associated with the polyol or DMAc had reacted with the remaining isocyanates. This data indicated the ideal timepoints to achieve 75% fractional isocyanate conversion assuming a PCL diol with an Mn of 1250 was chosen for the second addition.

### AAO_2/AAO_5/AAO_6

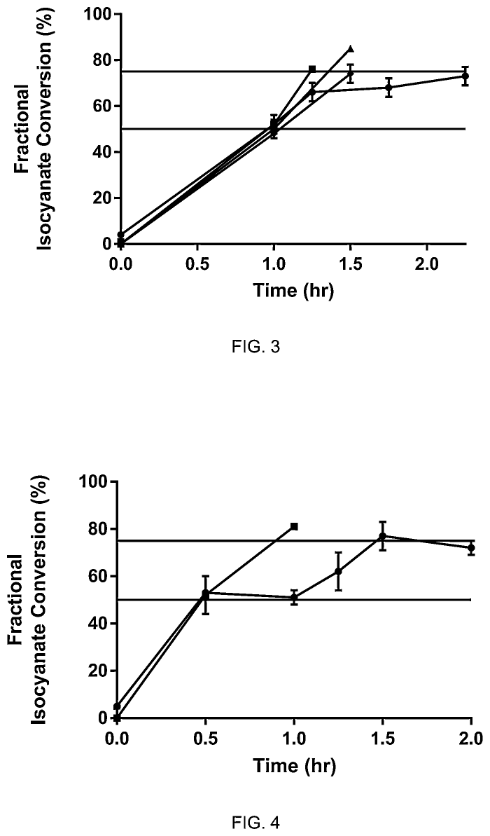

The kinetics depicted in FIG. 3 indicated a 50% conversion of LDI functionalities with PEG occurred after one hour of reaction time, at which point PCL was added, where 75% conversion was observed between 1.25 to 1.5 hours. Although a PCL triol, with a number average molecular weight of 900 was utilized, the fractional isocyanate conversion followed a similar trend as the diol in AAO_1, suggesting that the molecular weights of PEG and PCL, and the number of alcohol functionalities did not affect the fractional isocyanate conversion [D. Muscat, R. Adhikari, M. J. Tobin, S. McKnight, L. Wakeling, and B. Adhikari, “Effect of spatial distribution of wax and PEG-isocyanate on the morphology and hydrophobicity of starch films,” Carbohydr Polym, vol. 111, pp. 333-47, Oct. 13 2014].

### AAO_3

With reference to the black circles from FIG. 4, it was observed that 50% isocyanate conversion was achieved within 0.5 hours (PCL first addition), unlike PEG, which required 1.0 hour. This can be attributed to the increase in hydrogen bonding that occurs with ether functional groups in PEG, causing self-association of the oligomers and therefore reducing the reactivity of the hydroxyl groups, compared to the more hydrophobic PCL, a polymer with fewer hydrogen bonding domains [G. Raspoet, M. T. Nguyen, M. McGarraghy, and A. F. Hegarty, “The alcoholysis reaction of isocyanates giving urethanes: Evidence for a multimolecular mechanism,” The journal of organic chemistry, vol. 63, no. 20, pp. 6878-6885, 1998]. Following the 50% conversion, PEG was added dropwise, where 75% conversion was achieved after 0.5 hours. The black circle set of data suggested optimal times to reach 50 and 75% fractional isocyanate conversion, confirmed by the data in the black square timepoints. Overall, the total reaction time of AAO_3 was less than AAO_1 and AAO_2 due to the higher reactivity of PCL with isocyanates versus PEG during the first addition.

### AAO_4

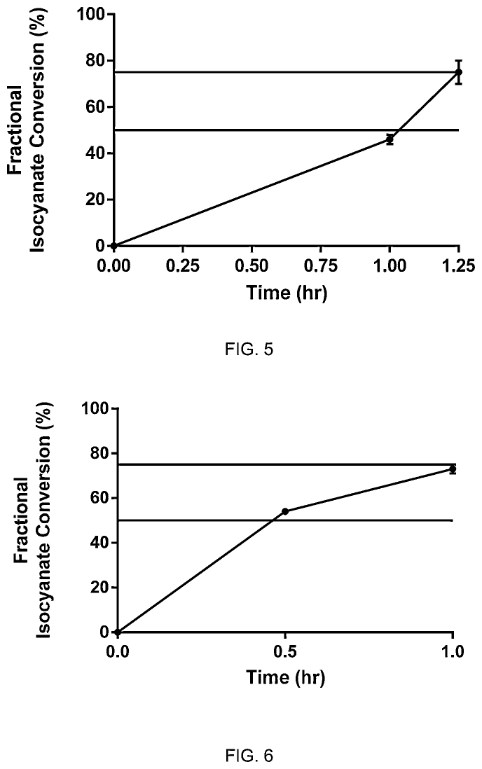

FIG. 5 indicated that 50% conversion of LDI functionalities with PEG occurred after an hour, at which point PCL was added where 75% conversion was observed at 1.25 hours. According to the results obtained from AAO_1, these chosen time points were selected due to a similar reaction scheme. In this case, AAO_4 differed from AAO_1 in that a PEG with an Mn of 2000 was used. Despite doubling the molecular weight of PEG, 50% conversion was still achieved at 1 hour.

### AAO_7

The kinetics shown in FIG. 6 indicated a 50% conversion of LDI functionalities during the first addition of PCL after 0.5 hours, at which time PEG was added where 75% conversion was observed after 1.0 hour. These reaction times were similar to the data observed for AAO_3 despite the number average molecular weight of PEG being 2000.

### AAO_8

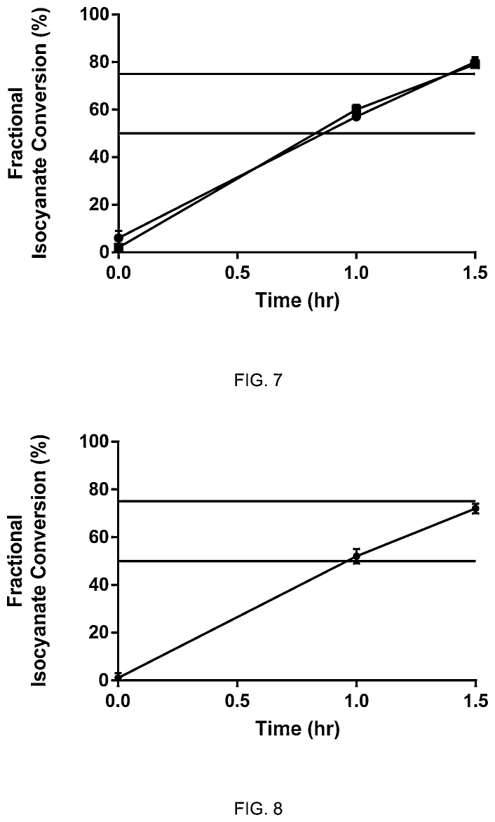

The kinetics shown in FIG. 7 indicated a 50% conversion of LDI functionalities with PEG occurred after an hour of the reaction, at which time PCL was added where 75% conversion was observed at 1.5 hours. AAO_8 followed a similar condition as AAO_2, in which a PCL triol with an Mn of 2000 was used.

### AAO_9

The kinetics from FIG. 8 indicated that 50% conversion of LDI functionalities with PEG occurred after one hour of the reaction, at which time PCL was added where 75% conversion was observed at 1.5 hours. The synthesis condition mimicked AAO_1, despite using a PEG with an Mn of 600.

In summary, PEGs with different Mn of 600, 1000 and 2000, did not display changes in the isocyanate kinetics since 50% conversion was achieved at an hour when the first polyol was added. During the second addition, the time required to obtain 75% conversion was similar between different AAO molecules (specifically AAO_1, 2, 4, 8, and 9) regardless of the number of hydroxyl groups on the PCL backbone or the number-average molecular weight. In the condition where PCL was the first polyol added to the reaction, such as AAO_3 and AAO_7, a shorter reaction time was needed to achieve the targeted 50% conversion (in which PEG was the second polyol added to obtain 75% conversion). These results validate a very producible and robust reaction protocol, which was then followed by end-capping all the residual isocyanate groups with DOPA, fake DOPA, or MeOH.

### Example 2: Physical and Chemical Characterizations of the Oligomers

### 2.1 Physical and Chemical Characteristics of the Oligomers

### 2.1.1 Solubility of Oligomers

Following the drying process, the oligomer was added to a scintillation vial and solubilized to a 20 wt. % solution. The solution was stirred on ice overnight, and the following day, the oligomer was assessed for water solubility [C. Y. Gong, S. Shi, P. W. Dong, B. Yang, X. R. Qi, G. Guo, Y. C. Gu, X. Zhao, Y. Q. Wei, and Z. Y. Qian, “Biodegradable in situ gel-forming controlled drug delivery system based on thermosensitive PCL-PEG-PCL hydrogel: part 1—Synthesis, characterization, and acute toxicity evaluation,” J Pharm Sci, vol. 98, no. 12, pp. 4684-94, December 2009; P. Patel, A. Mandal, V. Gote, D. Pal, and A. K. Mitra, “Thermosensitive hydrogel-based drug delivery system for sustained drug release,” Journal of Polymer Research, vol. 26, no. 6, p. 131, 2019; X. J. Loh, S. H. Goh, and J. Li, “Hydrolytic degradation and protein release studies of thermogelling polyurethane copolymers consisting of poly[(R)-3-hydroxybutyrate], poly(ethylene glycol), and poly(propylene glycol),” Biomaterials, vol. 28, no. 28, pp. 4113-23, October 2007].

### 2.1.2 Differential Scanning Calorimeter

Differential scanning calorimeter (DSC, Hitachi 7020 Thermal Analysis System) was used to study the thermal characteristics of the oligomers that were water-soluble according to Section 2.1.1. Approximately 10 mg of dried oligomer was added to an open aluminum pan. To start, under a nitrogen atmosphere, the sample was cooled to −90° C. at 10° C./min, then held for 30 minutes, and subsequently heated to 160° C. at 10° C./min. The sample was then cooled again and heated a second time. The results from the DSC curves were analyzed using NEXTA Standard Analysis software (Ver 2.0.0.5).

### 2.1.3 UV-Vis

The UV-Vis (Beckman DU 800 Spectrophotometer) was used as a quantitative assessment of the concentration of catechol species during the water washes. A calibration curve was created by solubilizing dopamine hydrochloride in Milli-Q water at concentrations between 0.001-0.025 wt. %.

### 2.1.4 Sodium Hydroxide Quantification

Sodium hydroxide (NaOH) was used as a reactant to enable the quantification of catechol species, as it oxidizes all catechol groups in the aqueous phase to form a coloured product. A calibration curve was generated by solubilizing dopamine hydrochloride in Milli-Q at concentrations between 0.001-0.05 wt. % with the addition of a drop of 10 N NaOH.

### 2.1.5 Nuclear Magnetic Resonance

The aqueous phase of the water washes was lyophilized. The remaining product post-lyophilization was dissolved in deuterated dimethylsulfoxide (d-DMSO) (Cambridge Isotope Laboratories, Inc.) and placed in 3 mm tubes (Norell Inc.) where ¹H-NMR and homonuclear correlation spectroscopy (COSY) spectra were obtained from the Bruker Avance III 400. The generated spectra were analyzed using MestReNova Version 14.1.0. Additionally, 1D and 2D NMR were utilized to characterize the final AAO products.

### 2.2 Purification

Following the reaction, it was critical to implement the water washes to ensure that these chemical groups did not contribute to toxicity concerns when evaluating the oligomers for their cell compatibility. The water-soluble species were characterized by UV-Vis, 1H-NMR, and a qualitative colourimetric assay during each water wash.

### 2.2.1 Quantitative Measure of Catechol Species in the Aqueous Phase

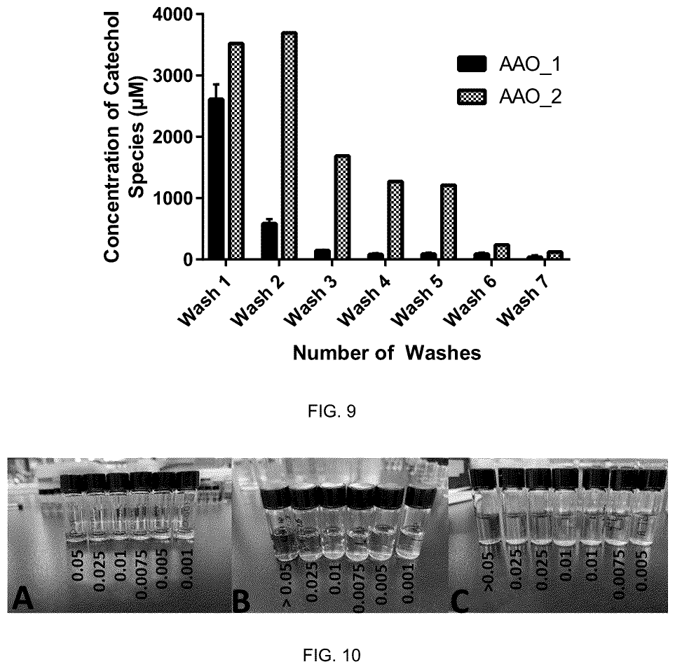

As shown in FIG. 9, the concentration of catechol species decreased with each subsequent water wash, thereby increasing the purity of the product. Amongst the two samples studied, seven washes were conducted to minimize the catechol species in the aqueous phase.

### 2.2.3 Qualitative Measure of Catechol Species in the Aqueous Phase

The addition of sodium hydroxide, as per methods described in Section 2.1.4, into the aqueous phase generated a coloured complex that could be detected qualitatively according to the concentration of catechol species as depicted in FIG. 10A. This was shown for the seven washes in FIG. 10B and FIG. 10C of AAO_1 and AAO_2, respectively, corresponding to the calibration curve. The results suggest a similar trend as the quantitative characterization in Section 2.2.1, where the largest concentration of catechol species was detected amongst the first few washes, while lower levels of catechol were detected in the latter washes.

### 2.2.4 Nuclear Magnetic Resonance

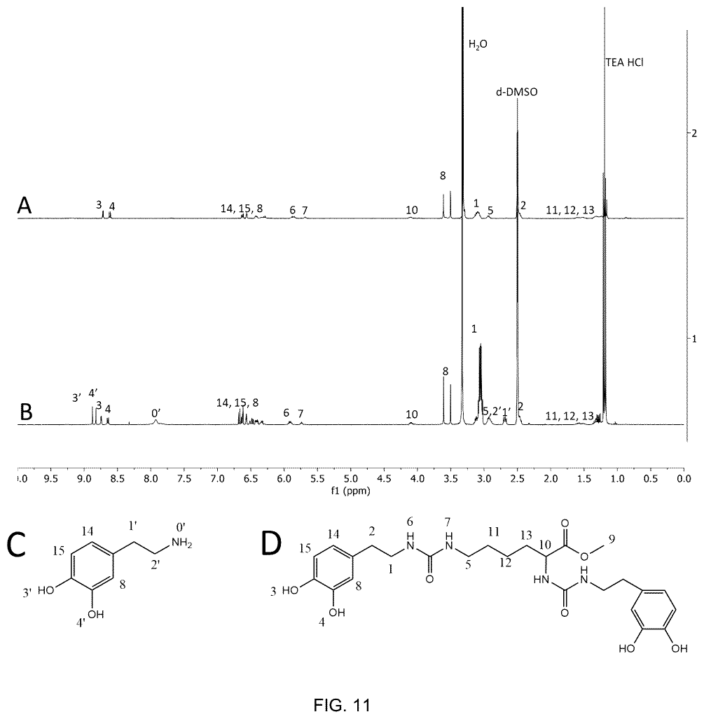

¹H-NMR confirmed that several water-soluble groups remained in the 1^{st} wash of the aqueous phase post-lyophilization. According to UV-Vis and the qualitative experiment, it was not surprising that unreacted catechol species remained as observed between 8.5-9.0 ppm on the NMR spectra shown in FIG. 11. Once dopamine had reacted, the alcohol groups on the catechol group shift upfield, resulting in two sets of similar peaks. The reacted dopamine peak (peak 2, —CH₂—CH₂—NHCONH—) correlates with the urea protons (peak 6) observed between 5.75-6.0 ppm, confirming that catechol dimers were formed due to a slight molar excess of LDI and DOPA. In addition, PEG, PCL, and urethane groups, typically observed at 3.5, 1.8, and 7.0-7.75 ppm respectively, were not seen, suggesting that the oligomer was not solubilized in the aqueous phase and remained in the organic layer. Additionally, the peak at 1.18 is denoted by triethylamine hydrochloride that is sparingly soluble in DMAc and filtered through the Buchner filtration setup. Later washes were analyzed for ¹H-NMR, but the similar peaks mentioned earlier were not detected due to the detection limit of the machine.

In summary, these multiple characterization techniques determined the post-processing necessary in order to purify the materials. By increasing the purity of the material, fewer variables are considered when evaluating the leached component of the phase-separated gel in Example 4.

### 2.3 Nuclear Magnetic Resonance

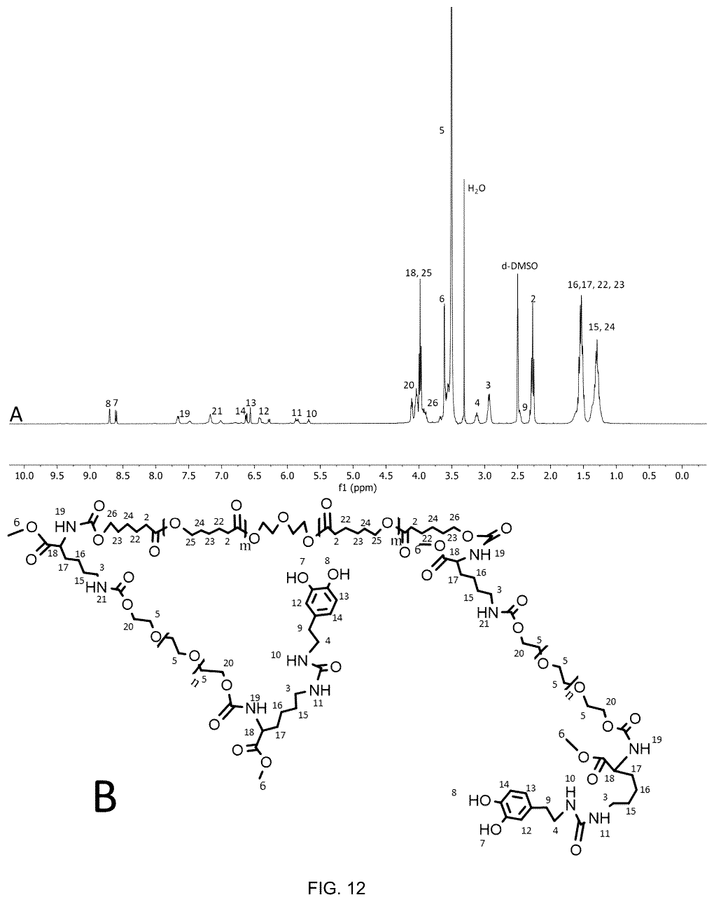

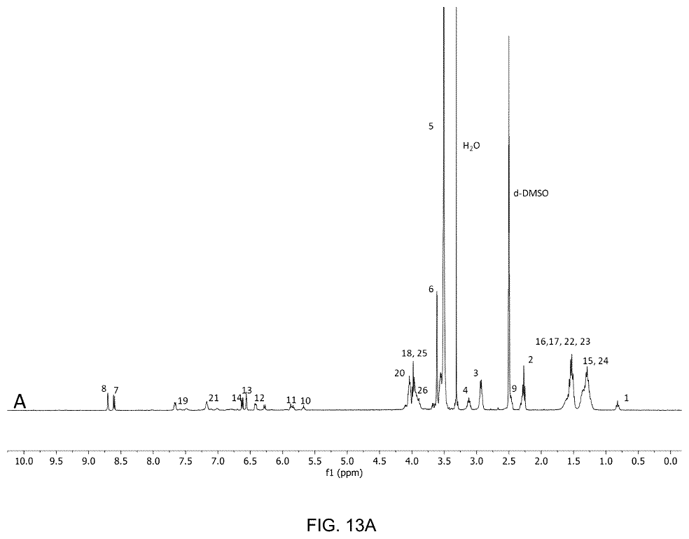

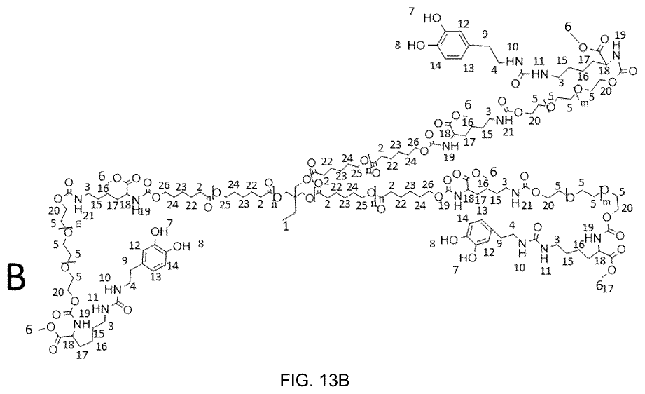

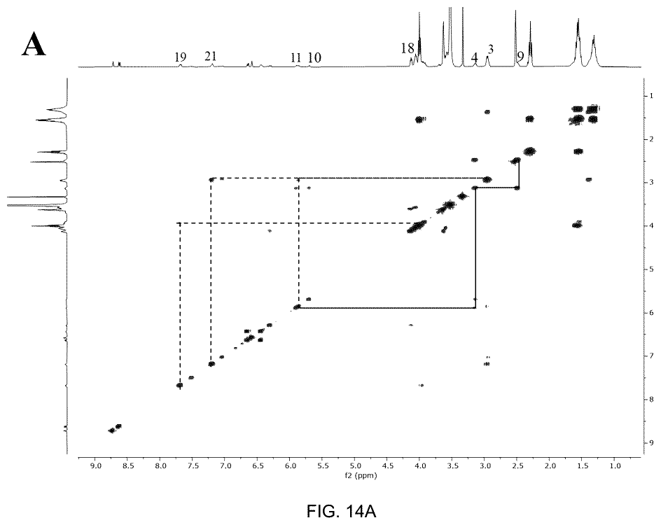

The nine synthesized oligomers were characterized by ¹H-NMR spectroscopy to determine the molecular weight of the structures and validate the synthesis strategy. In this Section, the spectra of AAO_1 and AAO_2 are shown in FIGS. 12 and 13 and are further discussed and characterized. The rationale for selecting these two oligomers relates specifically to the interesting gelation character of the AAO_1 and AAO_2 materials.

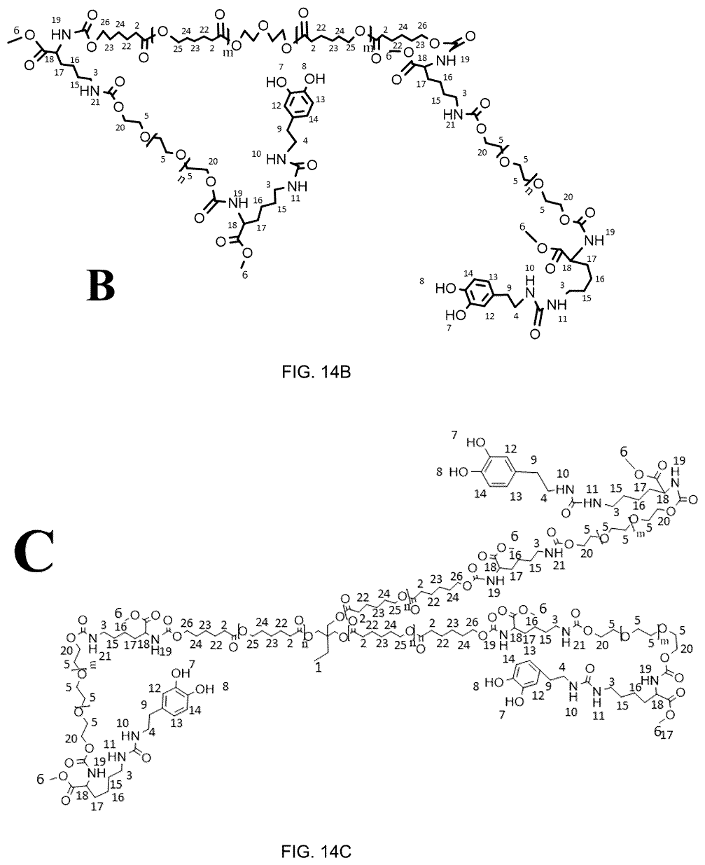

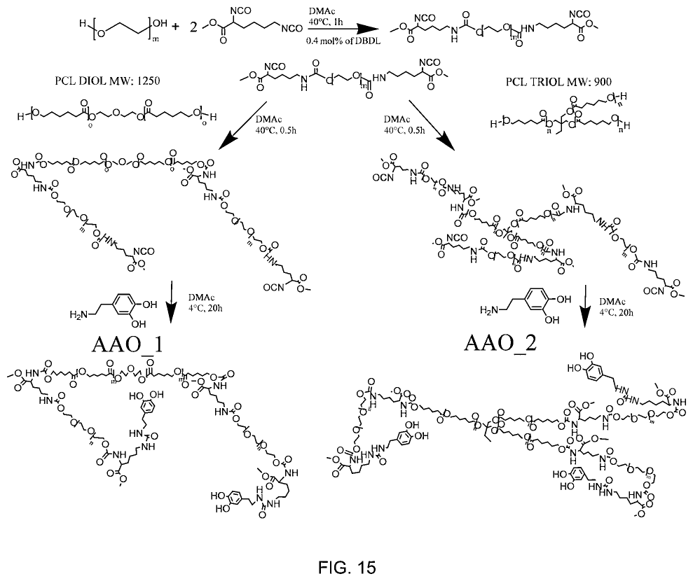

The structure was determined by identifying unique peaks for all the monomers. PEG and PCL have distinct peaks at 3.51 (—O—CH₂CH₂—O—) and 2.29 ppm (—O—CH₂—CH₂—CH₂—CH₂—CH₂—COO—), respectively, while the methyl peak on LDI is observed at 3.6 ppm. COSY NMR shown in FIG. 14A confirmed the peaks that are associated with dopamine and additionally the protons adjacent to the urethane and urea peaks which correspond to the LDI and DOPA. The urea peaks denoted as peaks 10 and 11 in FIG. 14A fall between 5.5-6.0 ppm with a correlation to the primary isocyanate at 2.9 ppm (NH—CH₂—CH₂—CH₂—CH₂—CH—) and the methylene group at 3.1 ppm on DOPA (—CH₂—CH₂—NH—CO—) referred to as peaks 4 and 3, respectively. According to the COSY analysis, the amine from dopamine reacted solely with the primary isocyanate on the LDI since there is no correlation between the urea peaks and the methine protons on LDI (NH—CH₂—CH₂—CH₂—CH₂—CH—) at 3.96 ppm. To further characterize the peaks on DOPA, peak 4 has an additional correlation with the adjacent methylene group on the dopamine at 2.5 ppm (—CH₂—CH₂—NH—CO—). This peak overlapped with the solvent peak from d-DMSO, thus the peak at 3.1 ppm was the unique peak to normalize all protons. The urethane groups were referred to as peaks 19 and 21 and correlated with secondary and primary isocyanates. The methine group, adjacent to the secondary isocyanate, has a de-shielding effect on the urethane, causing the protons to be slightly downfield compared to the methylene protons next to the primary isocyanate, which differentiates the urethane groups. When calculating for the number average molecular weight, the actual number of protons from PEG, PCL, LDI and DOPA were divided by the theoretical value, assuming no degree of oligomerization, as shown in Table 2. According to the actual and theoretical number-average molecular weight of AAO_1 and AAO_2, this synthesis pattern resulted in a small degree of oligomerization, indicating a quite successful synthesis and confirming the process shown in FIG. 15. Oligomerization would affect the PCL/PEG ratio, thus offsetting the solubility and the thermoresponsive properties. Some of the remaining oligomers (AAO_7 and AAO_8) had a higher degree of oligomerization, which potentially resulted from a faster addition rate of the first polyol to LDI. Lastly, in both the spectra for AAO_1 and AAO_2, no peaks correlated to any residual solvents and monomers, including DMAc, chloroform, ethyl ether, dopamine, and triethylamine hydrochloride, indicating a successful purification and drying process.

TABLE 2
Integration values of peaks in 1H-NMR of FIG. 12 and FIG. 13 of AAO_1
and AAO_2, respectively. The protons were normalized to the peak labelled with the asterisk
(peak 4), where the theoretical protons correspond to the ideal molar ratio of each monomer. The
remaining peaks in this table correspond to peak 5 on PEG at 3.51 ppm (O—CH₂—CH₂—O), peak 2
on PCL at 2.29 ppm (O—CH₂—CH₂—CH₂—CH₂—CH₂—COO), and lastly, peak 6 on LDI at 3.6
ppm (CH₃—O—CO—CH). The ratio column calculated the actual number of protons from ¹H-NMR
divided by the theoretical numbers for each oligomer in which the number-average molecular weight
can be calculated. These protons were analyzed by MestReNova Version 14.1.0.
Integrated Protons

| | AAO_1 | | | | AAO_2 | | | |
|---|---|---|---|---|---|---|---|---|
| Number | Actual | Theoretical | Ratio | ppm | Actual | Theoretical | Ratio | ppm |
| 1 | — | — | — | — | 3.3 | 3 | | 0.7 |
| 2 | 28.2 | 23.5 | $= \frac{28.2}{23.5}$ | 2.3 | 15.6 | 13.8 15.6 | $= \frac{15.6}{13.8}$ | 2.3 |
| 3 | 12.8 | 8 | | 2.8 | 13.0 | 12.0 | | 2.8 |
| 4* | 4* | 4 | =2 | 3.1 | 6* | 6 | =3 | 3.1 |
| 5 | 216 | 168 | $= \frac{216}{168} + 1$ | 3.5 | 287 | 252 | $= 2 * \frac{287}{252} + 1$ | 3.5 |
| 6 | 17.1 | 12 | $= 2 * \frac{17.1}{12} + 2$ | 3.6 | 19.7 | 18 | $= 4 * \frac{19.7}{18.} + 2$ | 3.6 |
| 7 | 1.9 | 2 | | 8.7 | 2.2 | 3 | | 8.7 |

| | | |
|---|---|---|
| Actual Mₙ | $= 212.21 * ( 2 * \frac{17.1}{12} + 2 ) + 1000 * ( \frac{216}{168} + 1 ) + \\ 189.21 * 2 + 1250 * ( \frac{28.2}{23.5} ) ∼ 5 , 225.4 g / mol$ | $= 212.21 * ( 4 * \frac{19.7}{18.} + 2 ) + 189.21 * 3 + \\ 1000 * ( 2 * \frac{287}{252} + 1 ) + 900 * ( \frac{15.6}{13.8} ) ∼ 6215.9 g / mol$ |
| Theoretical Mₙ | $212.21 * ( 2 * \frac{12}{12} + 2 ) + 1000 * ( \frac{168}{168} + 1 ) + \\ 189.21 * 2 + 1250 * ( \frac{23.5}{23.5} ) ∼ 4 , 477.08 g / mol$ | $= 212.21 * ( 4 * \frac{18}{18.} + 2 ) + 189.21 * 3 + \\ 1000 * ( 2 * \frac{252}{252} + 1 ) + 900 * ( \frac{13.8}{13.8} ) ∼ 6090.62 g / mol$ |

### 2.4 Solubility of Oligomer

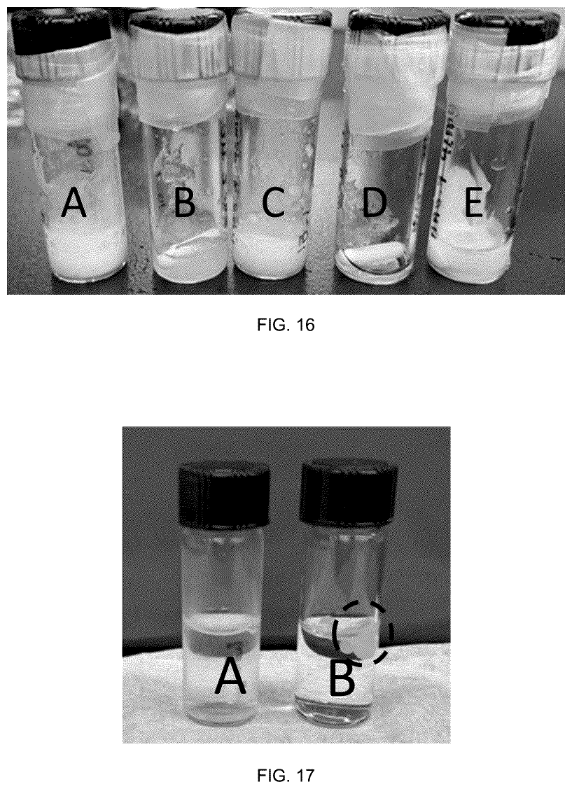

Post synthesis and purification, the oligomers were stirred overnight on ice, and on the following day, the oligomers were identified as either soluble, insoluble, or partially soluble, as shown in Table 3. Amongst the nine, AAO_1 and AAO_2/5/6 were completely water-soluble. FIG. 16 shows the remaining oligomers that were not water-soluble. AAO_4, 7, and 9 (depicted as A, C, and E from FIG. 16) were denoted as partially soluble since most of the material was soluble with residual oligomer that did not solubilize. The remaining AAO oligomers were not water-soluble, denoted as B and D in FIG. 16.

TABLE 3
A summary table of each oligomer and its respective number average molecular
weight, number of alcohol functionalities, the terminal end groups, and if each
oligomer is soluble. After overnight stirring, the oligomers were either soluble,
insoluble, or partially soluble, denoted as S, IS, or PS, respectively.
The PCL-PEG-PCL (AAO_3 and AAO_7) sequence is denoted with a “‡” meanwhile
PEG-PCL-PEG (AAO_1, AAO_2, AAO_4, AAO_5, AAO_6, AAO_8, and AAO_9)
sequence is denoted as a “*.”

| | PEG | | | PCL | | | End Termianl Group | | | Ratio | |
|---|---|---|---|---|---|---|---|---|---|---|---|
| | 600 - | 1000 - | 2000 - | 1250 - | 900 - | 2000 - | | Fake | | PCL/ | |
| Legend | Diol | Diol | Diol | Diol | Triol | Triol | DOPA | DOPA | MeOH | PEG | Soluble |
| AAO_1* | | X | | X | | | X | | | 0.67 | S |
| AAO_2* | | X | | | X | | X | | | 0.31 | S |
| AAO_3^{‡} | | X | | X | | | X | | | 2.05 | PS |
| AAO_4* | | | X | X | | | X | | | 0.38 | IS |
| AAO_5* | | X | | | X | | | X | | 0.31 | S |
| AAO_6* | | X | | | X | | | | X | 0.31 | S |
| AAO_7^{‡} | | | X | X | | | X | | | 1.04 | PS |
| AAO_8* | | X | | | | X | | | | 0.62 | IS |
| AAO_9* | X | | | | X | | X | | | 0.57 | PS |

### Example 3: Characterization of the Hydrogel

### 3.1 Characterization of the Hydrogel

### 3.1.1 Assessment of Thermoresponsive Properties

If the oligomers from Section 2.1.1 were water-soluble, they were added into a 1 mL syringe with an 18 G needle and injected into either a vial with PBS at 4° C. or 37° C. to evaluate its thermoresponsive properties.

### 3.1.2 UV-Vis

The UV-Vis (Beckman DU 800 Spectrophotometer) was utilized to determine the chemical crosslinking mechanisms of dopamine. The oligomers were solubilized in PBS, and the samples were scanned between 800 to 200 nm to measure the absorbance values. The UV-Vis was blanked to PBS without the addition of the adhesive.

### 3.1.3 Hydrogel Degradation

20 wt. % solutions of the two oligomers were prepared according to Section 2.1.1. A 0.5 borosilicate glass dram with 1 mL of simulated synovial fluid (SSF) was pre-weighed [K. L. Bertram, U. Banderali, P. Tailor, and R. J. Krawetz, “Ion channel expression and function in normal and osteoarthritic human synovial fluid progenitor cells,” Channels (Austin), vol. 10, no. 2, pp. 148-57, 2016]. The oligomer was loaded into a 1 mL syringe with an 18 G needle, injected into the pre-weighed dram vial, and the mass was recorded as the initial mass. At pre-determined time points, the dram vials were inverted to remove the solution, the wet mass of the gel was weighed, and lastly replenished with 1 mL of SSF. Two replicates were measured for each oligomer. The percent of wet mass was calculated according to Equation 2. The experiment was completed once with two replicates.

$$Wet Mass ( \% ) = 100 \% * ( \frac{( Mass of the {Gel}_{@ t = x} - Mass of the Empty Dram Vial )}{( Mass of the Gel and Fluid in the Dram {Vial}_{@ t = 0} - Mass of the Fluid in the Dram {Vial}_{@ t = 0} )} ) \qquad ( 2 )$$

### 3.1.4 Unconfined Compression Testing

20 wt. % solutions of each oligomer were prepared according to Section 2.1.1. Clear nail polish was applied onto one side of an 8×8 mm Borosilicate Cloning Cylinders (Pyrex, Corning) and glued onto a glass slide. Once dried, the sample was placed into a 35×10 mm Polystyrene Petri Dish (Falcon). 5 mL of PBS was added, and the sample was stored in a 37° C. oven. The oligomer was added into a 1 mL syringe, where approximately 250 μL was injected through an 18 G needle into the cylinders and stored in a 37° C. incubator. The gels were incubated for three days, after which the gels would be physically stable enough to handle and under compressive loading. The gels were gently removed from the holders by a 30 G needle (Precision Glide). Before evaluating the gels, the thickness and diameter were captured by Navitar using a Sony XCD-X710 Camera and the CellScale Biaxial Tester respectively, meanwhile, the measurements were characterized on ImageJ v1.51 by pixel counting to a referenced glass slide. The unconfined compression was completed on the TestResources 840 Series Frame & E Actuator apparatus and the Test Builder software using a WF-5 G load cell and Teflon platen, where the gel was evaluated in the Petri Dish that had been stored with 37° C. PBS. The gels were pre-loaded to 0.01 N, pre-conditions for five cycles at 10% of the initial thickness, and lastly, compression was applied at a constant rate of 3% s⁻¹ (relative to the gel's thickness) to a total strain of 75%. Stress and strain were calculated according to Equations 3 and 4, respectively, where WF5 in grams was reported from the Test Builders software. The compressive modulus was calculated using the linear region between the stress-strain curve under 20-30% strain. Six replicates were completed for each oligomer.

$$σ [ \frac{N}{{m}^{2}} ] = ( \frac{WF 5 * 9.81}{Area * 1000} ) \qquad ( 3 )$$

$$ε ( \% ) = 100 \% * ( \frac{Δ L}{Thickness} ) \qquad ( 4 )$$

### 3.2 Assessment of Thermoresponseness

Desirable properties for an injectable thermoresponsive oligomer would include a flowable solution at low temperatures (for the purpose of these studies, this was defined as 4° C.) that can be injected through an 18 G needle; however, at a physiological temperature of 37° C., the solution would be in a gel-like state.

AAO_1 and AAO_2 were assessed for this property by solubilizing the oligomer with PBS to a final concentration of 20 wt. %. Shown in FIG. 17A and FIG. 17B is the oligomer when injected into a vial containing PBS at 4° C. versus 37° C., respectively. At 4° C., the oligomer remained suspended in solution, meanwhile, at 37° C., there is a distinct formation of a phase-separated gel shown in FIG. 17B outlined in a dotted black circle implying thermoresponsive properties. Both oligomers showed similar responses, but this photo represented AAO_2 specifically. Samples prepared at 25 to 45 wt. % of oligomers in solution were soluble; however, they could not form flowable solutions that were injectable when loaded into a 1 mL syringe with an 18-gauge needle due to their inherent viscosity. Thus, 20 wt. % was used for all hydrogel characterization in the ongoing work of all the Examples.

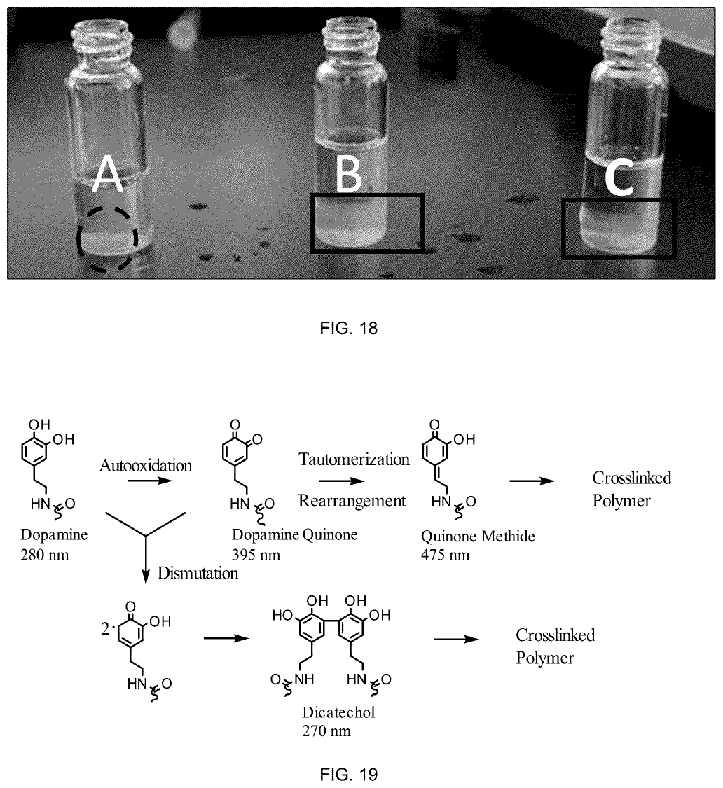

AAO_5 and AAO_6 with terminal domains of 3-4-dimethoxyphenethylamine and methanol, respectively, were assessed for their thermoresponsive property. The two oligomers were dissolved at 20 wt. % and injected into a PBS solution at 37° C. FIG. 18B and FIG. 18C showed that a diffuse translucent/opaque suspended material was formed rather than the well-defined gel (AAO_2 depicted in FIG. 18A) at the bottom of the vial outlined in an black box.

As vials B and C in FIG. 18B and FIG. 18C, respectively, were tilted from side to side, the weakly formed gel flowed in the respective directions. Following a quick mix on the Vortex Mixer, the gel broke apart, suggesting weak intermolecular forces occurred between oligomer chains. For AAO_2, the gel did not flow and remained intact. This observation suggests that the DOPA hydroxyls are important elements for stabilizing the reverse phase-separated gel. MeOH terminated domains (sample B in FIG. 18) comprised solely of Van der Waals interaction between the methoxy groups. Additionally, this oligomer includes only urethane functionalities. Comparing F-DOPA and DOPA, both contain urea functionalities that link together the isocyanate, as shown in Table 4. The hydrogen bonding of the two N—H groups on urea is stronger than the single N—H group on urethane, resulting in a weaker gel network. Similar to the methanol terminated domain, the F-DOPA comprises of Van der Waals forces from the methoxy groups of the benzene ring, along with the additional pi-pi stacking capacity from that ring structure, potentially resulting in stronger networks. Though, with a DOPA functionality, hydrogen bonding and the pi-pi interaction between the catechol domains provide additional stability of the 3D matrix, resulting in a well-formed gel for AAO_2.

TABLE 4
A schematic of the terminal domains of AAO_2, AAO_5, and
AAO_6 with reference to DOPA, 3-4-dimethoxyphenethylamine,
and methanol, respectively.
AAO_2 (DOPA)

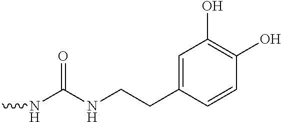

AAO_5 (3-4-dimethoxyphenethylamine)

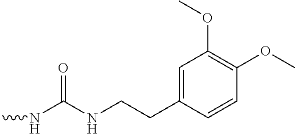

AAO_6 (methanol)

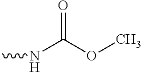

### 3.3 UV-Vis

A key objective for these oligomers is sufficient chemical crosslinking without the addition of chelating or oxidizing agents in order to stabilize the gel. UV-Vis can confirm these self-crosslinking interactions. A mechanism for the chemical crosslinking of AAOs is depicted in FIG. 19 [B. P. Lee, J. L. Dalsin, and P. B. Messersmith, “Synthesis and gelation of DOPA-modified poly(ethylene glycol) hydrogels,” Biomacromolecules, vol. 3, no. 5, pp. 1038-47, September-October 2002; P. Sun, J. Wang, X. Yao, Y. Peng, X. Tu, P. Du, Z. Zheng, and X. Wang, “Facile preparation of mussel-inspired polyurethane hydrogel and its rapid curing behavior,” ACS Appl Mater Interfaces, vol. 6, no. 15, pp. 12495-504, Aug. 13 2014].

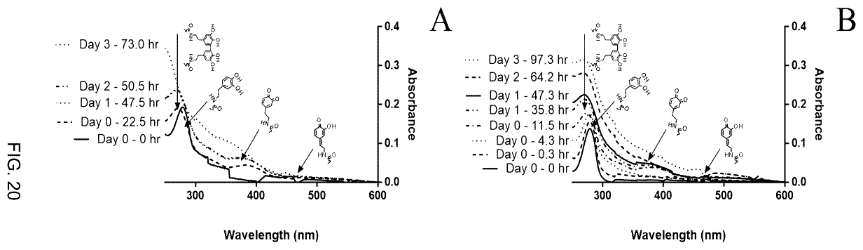

AAO_1 and AAO_2 were diluted to a concentration in which it was completely soluble, where the intermediate catechol products had unique wavelengths between 800 to 200 nm. The catechol domains appeared at 280 nm, as shown in FIG. 20A and FIG. 20B. As dopamine oxidized via the autoxidation process, dopamine quinone was detected by UV-Vis at 395 nm. This occurs as early as 11.5 hours post the start of the crosslinking process. The absorbance peak at 395 nm constantly increases until the 47^{th} hour. During the autoxidation process, the first step entailed the oxidation of catechol from oxygen, and according to the redox potential of catechol/o-semiquinone and O₂/O₂⁻, these have different reaction potentials of 530 and −155 mV, respectively. The equilibrium will not favour the catechol reaction since higher redox potential results in a greater chance of being reduced, resulting in a slow oxidation process (also known as a higher oxidizing agent) [J. Yang, M. A. Cohen Stuart, and M. Kamperman, “Jack of all trades: versatile catechol crosslinking mechanisms,” Chem Soc Rev, vol. 43, no. 24, pp. 8271-98, Dec. 21 2014; P. Sun, J. Wang, X. Yao, Y. Peng, X. Tu, P. Du, Z. Zheng, and X. Wang, “Facile preparation of mussel-inspired polyurethane hydrogel and its rapid curing behavior,” ACS Appl Mater Interfaces, vol. 6, no. 15, pp. 12495-504, Aug. 13 2014]. Once dopamine quinone is formed, it can undergo two crosslinking pathways that form additional intermediate products. One product is quinone methide, in which a tautomerization process occurs. This can be seen in this system at 475 nm. The second intermediate product is a semiquinone radical formed between the dopamine and dopamine-quinone, referred to as the dismutation process. When the two radicals react together, di-catechol crosslinked species appear at 270 nm in the UV spectrum. This happened as early as 11.5 hours post-gelation, where the dopamine (typically seen at 280) has shifted to 270 nm due to the di-DOPA crosslinking by the aryloxy radical coupling [J. Yang, M. A. Cohen Stuart, and M. Kamperman, “Jack of all trades: versatile catechol crosslinking mechanisms,” Chem Soc Rev, vol. 43, no. 24, pp. 8271-98, Dec. 21 2014; P. Sun, J. Wang, X. Yao, Y. Peng, X. Tu, P. Du, Z. Zheng, and X. Wang, “Facile preparation of mussel-inspired polyurethane hydrogel and its rapid curing behavior,” ACS Appl Mater Interfaces, vol. 6, no. 15, pp. 12495-504, Aug. 13 2014].

There is a general lack of consensus in the literature of the specific mechanisms that define how these intermediate products can further crosslink into a polymerized network. As time passed, the absorbance of this peak increased, evidencing a higher degree of crosslinking. This reaction was observed under PBS and not in a highly oxidative environment. Thus, this would simulate a slower chemical crosslinking.

### 3.4 Differential Scanning Calorimetry

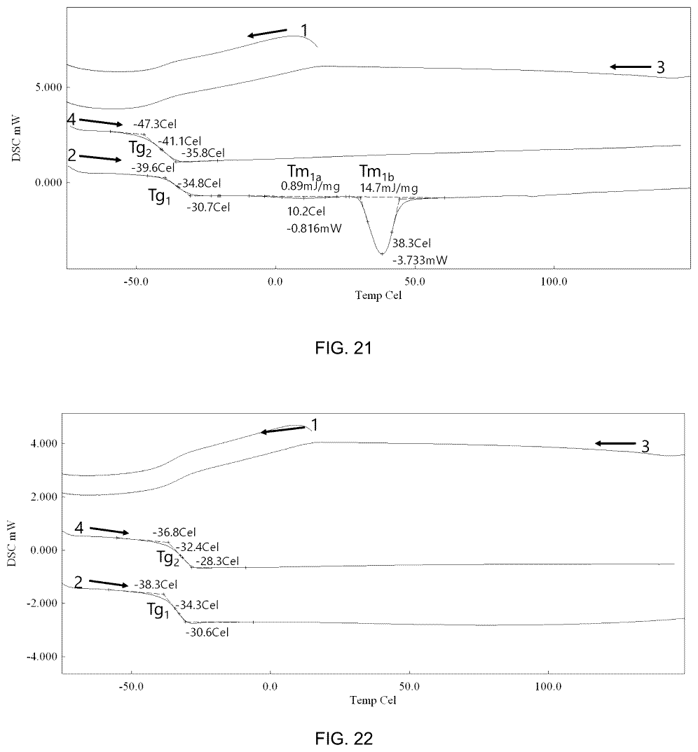

The thermograms of AAO_1 and AAO_2 are shown in FIGS. 21 and 22, respectively, while the analyzed results are reported in Table 5. Polyurethanes are traditionally defined by their unique configuration of soft and hard segments. Examples of soft segments include polyesters and polyethers, whereas the hard domains are attributed to the isocyanate-containing regions. The two segments can be immiscible, resulting in two distinct thermal characteristics between both domains. For example, two glass transition temperatures and/or two melt temperatures [J. P. Santerre, K. Woodhouse, G. Laroche, and R. S. Labow, “Understanding the biodegradation of polyurethanes: from classical implants to tissue engineering materials,” Biomaterials, vol. 26, no. 35, pp. 7457-70, December 2005].

For the current materials, one unique glass transition temperature (T_{g}) was observed in these thermograms. The T_{g} of PCL monomer is between −60 to −64° C. as shown in Table 5, though the results for the AAO materials indicated a higher T_{g}, attributed by partial mixing between the soft and hard domains [T. W. Son, D. W. Lee, and S. K. Lim, “Thermal and phase behavior of polyurethane based on chain extender, 2, 2-bis-[4-(2-hydroxyethoxy) phenyl] propane,” Polymer journal, vol. 31, no. 7, pp. 563-568, 1999; P. Krol, “Synthesis methods, chemical structures and phase structures of linear polyurethanes. Properties and applications of linear polyurethanes in polyurethane elastomers, copolymers and ionomers,” Progress in materials science, vol. 52, no. 6, pp. 915-1015, 2007; L. Yu, L. Zhou, M. Ding, J. Li, H. Tan, Q. Fu, and X. He, “Synthesis and characterization of novel biodegradable folate conjugated polyurethanes,” Journal of colloid and interface science, vol. 358, no. 2, pp. 376-383, 2011]. Additionally, the T_{g} for AAO_2 was higher than AAO_1, without wishing to be bound by a theory, possibly due to a branched PCL structure that reduced chain mobility and most likely increased phase mixing between the soft and hard domains [A. Guney and N. Hasirci, “Properties and phase segregation of crosslinked PCL-based polyurethanes,” Journal of Applied Polymer Science, vol. 131, no. 1, 2014]. A crystalline melt temperature (Tₘ) was only seen for AAO_1, indicating a semi-crystalline structure. However, this was not the case for AAO_2 which resulted in an amorphous oligomer, potentially due to the disruptive effect from the branched chains and the rigidity and bulkiness from the catechol groups, which would further restrict the chain mobility to enable packing and eventual crystallization [E. Filippidi, T. R. Cristiani, C. D. Eisenbach, J. H. Waite, J. N. Israelachvili, B. K. Ahn, and M. T. Valentine, “Toughening elastomers using mussel-inspired iron-catechol complexes,” Science, vol. 358, no. 6362, pp. 502-505, 2017; N. Jia, Q. He, J. Sun, G. Xia, and R. Song, “Crystallization behavior and electroactive properties of PVDF, P (VDF-TrFE) and their blend films,” Polymer Testing, vol. 57, pp. 302-306, 2017].

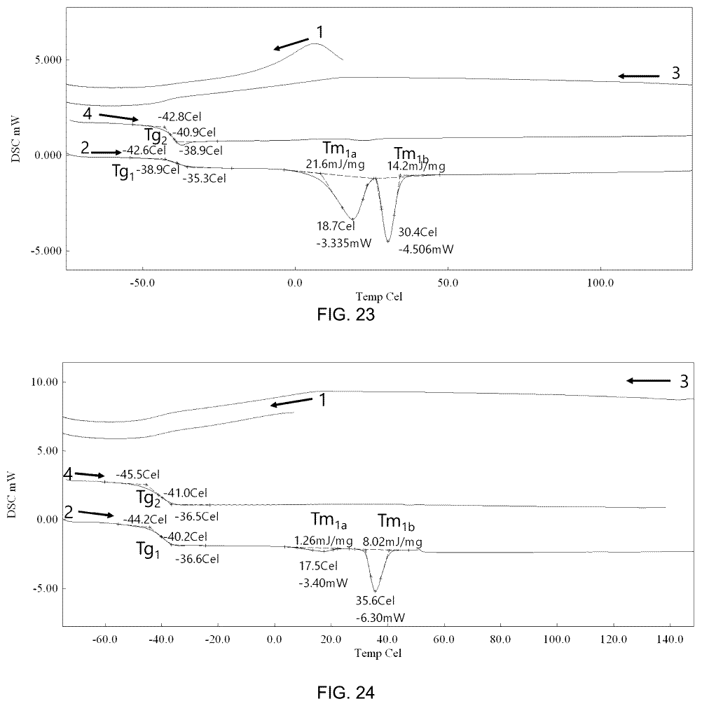

The influence of the DOPA function is featured when comparing the thermogram between AAO_2 with that of AAO_6 in FIG. 23 (both materials had similar reagents, however, AAO_2 comprised of DOPA terminal groups while AAO_6 was MeOH terminated) AAO_6 displayed two broad Tₘ endotherms attributed to the melting of PCL 900 and influenced by the PEG moieties confirming that the branched oligomers were restricted in their movement by the catechol. During the first heat cycle of AAO_1, the first melt temperature (Tₘ₁ₐ) is attributed to the melt of PCL, this was confirmed by comparison to the PCL monomer itself shown in FIG. 21. Following the cooling cycle and reheating process, there were no observed crystallization and melt temperatures. This suggests that a slower cooling would allow for the recrystallization of these regions [B. R. Barrioni, S. M. de Carvalho, R. L. Oréfice, A. A. R. de Oliveira, and M. de Magalhães Pereira, “Synthesis and characterization of biodegradable polyurethane films based on HDI with hydrolyzable crosslinked bonds and a homogeneous structure for biomedical applications,” Materials Science and Engineering: C, vol. 52, pp. 22-30, 2015]. To validate this hypothesis, a previously tested AAO_1 sample was placed in the freezer for two days to provide sufficient time for the soft segment to recrystallize. The result is shown in FIG. 24, confirming similar peaks as FIG. 21. Lastly, on the second heating cycle, there was a slight decrease in T_{g} for AAO_1 and AAO_6, possibly attributed to the absence of crystallization during the second cooling cycle; a reduction in crystalline domain suggests an increase in molecular mobility.

TABLE 5
The T_{g} and Tₘ (° C.) of AAO_1 and AAO_2 measured using NEXTA Standard
Analysis software (Ver 2.0.0.5). Additionally, PEG 1000, PCL 1250, and PCL 900 monomers were
included for comparison. Results were obtained from completing triplicates for AAO_1 and AAO_2
(n = 3), where the standard deviation was calculated.

| Sample | Tg₁ (° C.) | Tg₂ (° C.) | Tm₁ₐ (° C.) | $Δ {H}_{m 1 a} ( \frac{mJ}{mg} )$ | Tm_{1b} (° C.) | $Δ {H}_{m 1 b} ( \frac{mJ}{mg} )$ |
|---|---|---|---|---|---|---|
| AAO_1 | −36.7 ± 1.7 | −41.3 ± 0.4 | 15.8 ± 7.6 | 0.71 ± 0.2 | 35.9 ± 2.1 | 12.5 ± 2.0 |
| AAO_2 | −32.7 ± 1.1 | −34.5 ± 0.7 | ND | ND | ND | ND |
| AAO_6 | −38.9 | −40.9 | 18.7 | 21.6 | 30.4 | 14.2 |
| PEG 1000 | ND | ND | ND | ND | 36.4 | 122 |
| PCL 1250 | −64.3 | −62.1 | 10.8 | 30.2 | 41.5 | 25.4 |
| PCL 900 | −60.8 | −62.3 | 15.5 | 13.0 | 46.0 | 74.4 |

Abbreviations:
Tg₁, the glass transition temperature for the first heating cycle;
Tg₂, the glass transition for the second heating cycle;
Tm₁ₐ, the first melt temperature that appeared in the first heating cycle;
ΔHₘ₁ₐ, the corresponding heat of fusion from Tm₁ₐ [very small but detected];
Tm_{1b}; the second melt temperature that appeared in the first heating cycle;
ΔH_{m1b}; the corresponding heat of fusion from Tm_{1b}, no data; ND.

### 3.5 In Vitro Degradation

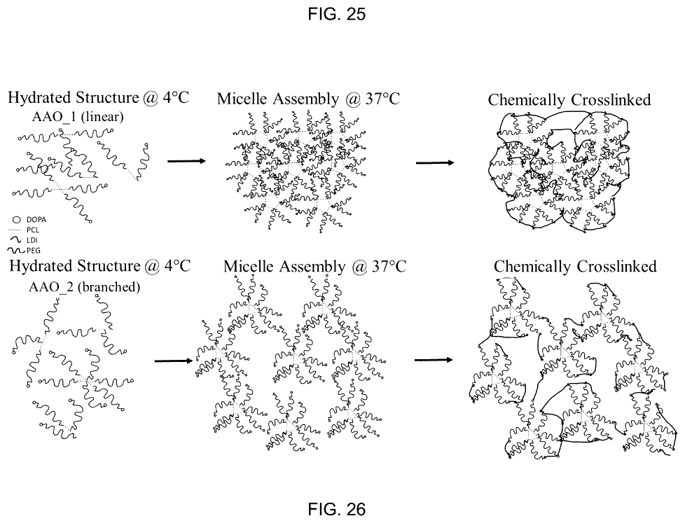

The degradation/swelling kinetics were characterized by weighing the wet mass over 21 days in which the phase-separated gel was submerged in 1 mL of simulated synovial fluid. All the data points were normalized to the initial mass injected into the dram vial, where FIG. 25 illustrates the in vitro swelling/degradation effects. Over 21 days, AAO_1 and AAO_2 remained stable at approximately 68% and 100% of their original wet mass, respectively. AAO_2 displayed a swelling effect attributed to a higher PEG percentage, as shown in Table 6. The percent of each monomer was calculated according to the number of protons from ¹H-NMR divided by Mn in Section 2.3.

Based on these results, physical and chemical crosslinking mechanisms for AAO_1 and AAO_2 are shown in FIG. 26. The branched polymer exhibits a higher degree of steric hindrance than the linear polymer, enabling greater stability in the first hours before DOPA interactions begin to crosslink. However, the micelles of AAO_2 cannot effectively pack together, which may have contributed to the higher degree of swelling since PEG has a high affinity for water. Conversely, for AAO_1, since there is a higher percent of PCL while PEG content is lowered, the micelles pack closer together, resulting in less swelling. Any remaining oligomer that did not aggregate into the bulk will be eluted into the simulated synovial fluid resulting in a decrease in weight as observed in the first day, particularly for AAO_1. According to the literature, in vitro degradation experiments of polyurethanes utilize PEG and PCL as the soft segments suggest a slow hydrolytic degradation profile. The delay is due to PCL's hydrophobicity, preventing the random hydrolytic cleavage of the ester linkages. Additionally, hydrogen bonding will be present between the soft and hard segments since only one glass transition temperature was observed, as outlined in the DSC data above. This will shield the ester groups from rapid hydrolysis and enhance the degradation profile [J. P. Santerre, K. Woodhouse, G. Laroche, and R. S. Labow, “Understanding the biodegradation of polyurethanes: from classical implants to tissue engineering materials,” Biomaterials, vol. 26, no. 35, pp. 7457-70, December 2005; J. P. Santerre, R. S. Labow, D. G. Duguay, D. Erfle, and G. A. Adams, “Biodegradation evaluation of polyether and polyester-urethanes with oxidative and hydrolytic enzymes,” J Biomed Mater Res, vol. 28, no. 10, pp. 1187-99, October 1994]. Lastly, the addition of terminal catechol groups introduced chemical crosslinking between the oligomers as characterized in Section 3.3, which, without being bound by a theory, may provide a further barrier for these ester and urethane domains to undergo rapid hydrolysis. In summary, this experiment provides evidence that the gel can remain stable for weeks in simulated synovial fluid.

TABLE 6
A percent composition breakdown of the monomers for AAO_1 and
AAO_2 calculated from ¹H-NMR in Section 2.3. The values were
calculated by dividing the number of protons for each monomer
by the number-average molecular weight for each respective oligomer.

| Monomers | AAO_1 (linear) | AAO_2 (branched) |
|---|---|---|
| PEG | 43.7% | 52.7% |
| PCL | 29.3% | 16.4% |
| LDI | 19.7% | 21.8% |
| DOPA | 7.2% | 9.1% |

### 3.6 Unconfined Compression Testing

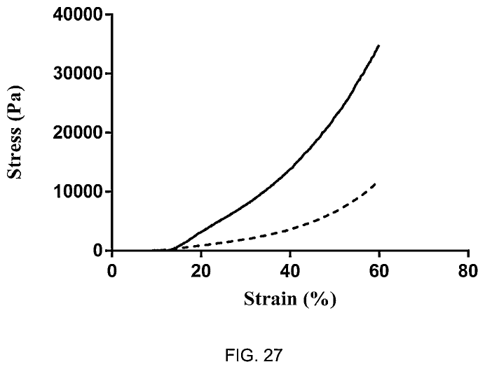

Unconfined compression testing is a method of mechanical characterization that can be used to evaluate the compressive moduli of the phase-separated gels [D. A. Prince, I. J. Villamagna, A. Borecki, F. Beier, J. R. de Bruyn, M. Hurtig, and E. R. Gillies, “Thermoresponsive and Covalently Cross-Linkable Hydrogels for Intra-Articular Drug Delivery,” ACS Applied Bio Materials, vol. 2, no. 8, pp. 3498-3507, 2019; T. Hao, N. Wen, J.-K. Cao, H.-B. Wang, S.-H. Lü, T. Liu, Q.-X. Lin, C.-M. Duan, and C.-Y. Wang, “The support of matrix accumulation and the promotion of sheep articular cartilage defects repair in vivo by chitosan hydrogels,” Osteoarthritis and Cartilage, vol. 18, no. 2, pp. 257-265, 2010]. One sample recording of each gel was plotted on a strain (%) versus stress curve, shown in FIG. 27. Before the evaluation, the gels were incubated at 37° C. for three days to maximize the chemical crosslinking between the catechol domains validated in Section 3.3. The compressive moduli reported in Table 6 was calculated based on the linear region between 20-30% strain. There was a statistically significant in the compressive modulus of AAO_1 to AAO_2 of specifically ten times higher. Although the DOPA content between the two oligomers had comparable values shown in Table 6, the major difference between the two oligomers is the overall structure, one (AAO_1) being linear with condensed hydrophobic domains while the other was branched (AAO_2) with a more open structure. Since AAO_2 exhibited a higher degree of swelling shown in FIG. 25, which resulted in a less dense network, a lower compressive modulus was recorded compared to AAO_1. Additionally, the proposed mechanisms presented in FIG. 26 of the branched network show a reduced packing density between the micelles due to the steric hindrance of the branched chains. Lastly, the DSC data indicated that AAO_2 was an amorphous polymer, while AAO_1 had semi-crystalline domains, resulting in an increase in compressive moduli for the latter due to the packing and immobilizing of the soft segments and/or PCL/urethane domains forming crystalline regions [M. Szycher, “Structure-property relations in polyurethanes,” Szycher's handbook of polyurethanes, vol. 37, 2012; S. Agarwal and C. Speyerer, “Degradable blends of semi-crystalline and amorphous branched poly (caprolactone): Effect of microstructure on blend properties,” Polymer, vol. 51, no. 5, pp. 1024-1032, 2010]. By comparing the compressive moduli of the meniscus and articular cartilage to AAO_1 and AAO_2, AAO_1 resembled values similar to the meniscus (see Table 7 below), suggesting a potential injection site for the gel to reside in.

TABLE 7
The compressive moduli of AAO_1 and AAO_2 of six replicates
for each oligomer. Compressive moduli of the meniscus and cartilage
are enclosed for reference purposes [source: H. N. Chia and M. L.
Hull, “Compressive moduli of the human medial meniscus in the
axial and radial directions at equilibrium and at a physiological strain
rate,” J Orthop Res, vol. 26, no. 7, pp. 951-6, July 2008
and R. K. Korhonen, M. S. Laasanen, J. Toyras, J. Rieppo, J. Hirvonen,
H. J. Helminen, and J. S. Jurvelin, “Comparison of the equilibrium
response of articular cartilage in unconfined compression, confined
compression and indentation,” J Biomech, vol. 35, no. 7, pp. 903-9,
July 2002, respectively]. Statistical significance was observed as p < 0.01
signified by ** with comparison to AAO_2.

| | Compressive Modulus | |
|---|---|---|
| Tested Materials | (kPa) | |
| AAO_1 | 44.8 ± 16.0 | kPa** |
| AAO_2 | 3.81 ± 2.65 | kPa |
| Meniscus | 37.3 ± 3 | kPa |
| Patella Cartilage | 570 ± 170 | kPa |

Their unique solubility and thermoresponsive characters make AAO_1 and AAO_2 particularly suitable for osteoarthritis (OA) applications.

The thermal characteristics were defined from the DSC results, suggesting that AAO_1 comprises of semi-crystalline domains, while AAO_2 is completely amorphous due to the unique combination of its branching structure and DOPA functionalities that prevented chain mobility from enabling assembly between the chains. In vitro stability studies of the two gels showed that AAO_2 swelled more due to the increase in PEG content when compared to AAO_1. This (AAO_2) increased structure with water appears to have stabilized the system within the first 24 hours before the DOPA crosslinking stabilized the materials. AAO_1, on the other hand, loses wet mass within 24 hours but then is stabilized for the remaining three weeks of the study, showing greater mechanical character than AAO_2. Both gels were stable for the 21 days at 37° C. in SSF solution. The UV-Vis data confirmed that catechol domains participate in chemical crosslinking, in which the quinone methide, di-catechols, and dopamine quinone were observed. These results defined the duration that the gels were incubated for before assessing for the compressive moduli to maximize catechol crosslinking. The compressive moduli of AAO_1 resembled similar values as that of the meniscus, due partly to the oligomer being inherently semi-crystalline, increasing the mechanical properties. At the same time, weaker gels were observed by AAO_2 due to a higher degree of swelling (10% increase with respect to the original dry mass) and potentially steric hindrance between oligomer chains, preventing densely packed micelles. When considering the injection site for future experiments, the meniscus is a potential option for the phase-separated gel to reside in. These experiments show encouraging physical, chemical, and mechanical properties for potential drug delivery vehicles for OA.

### Example 4: Evaluation of a Hydrogel's Biocompatibility

### 1st Biocompatibility Study

AAO_1 and AAO_2 were added into a separate autoclaved 1.0 Dram vial (borosilicate glass, VWR) along with an autoclaved stir-bar and sterile PBS. These precursors were solubilized to a 20 wt. % solution and stirred overnight at 4° C. The oligomer was loaded into a 1 mL sterile syringe with an 18 G needle and injected into another autoclaved dram vial with cell culture media at 37° C. Each condition comprised of a 1:80 ratio between the gel and cell culture media. Following a 24-hour incubation between the gel and cell culture media, the media was sterile filtered through a 0.22 μm filter (Millex-GV Syringe Filters, Polyvinylidene difluoride Durapore Membrane), and stored at −20° C. until further use.

### 2nd Biocompatibility Study

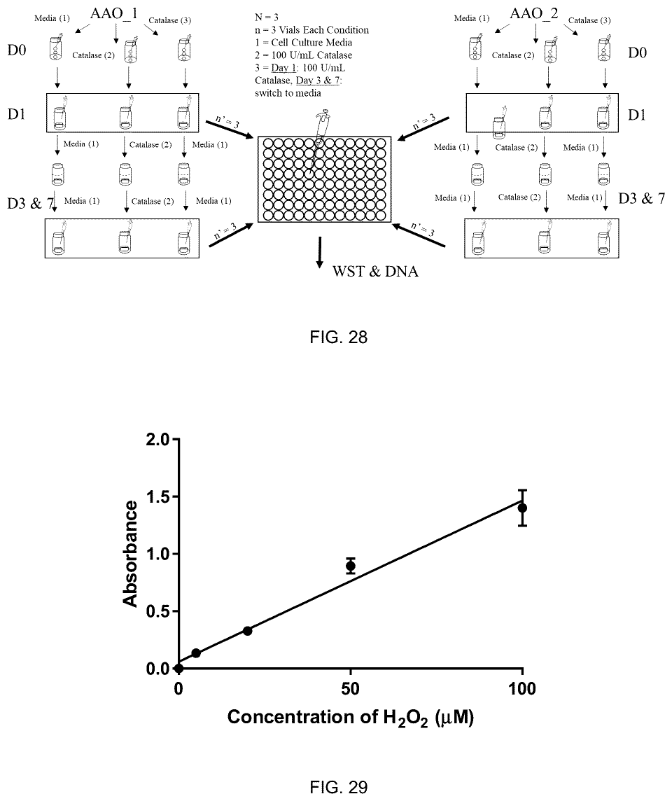

AAO_1 and AAO_2 were added into separate autoclaved 1.0-dram vial (borosilicate glass, VWR) along with an autoclaved stir-bar and sterile PBS. These precursors were solubilized to a 20 wt. % solution and stirred overnight at 4° C. The oligomer was loaded into a 1 mL sterile syringe with an 18 G needle and injected into another autoclaved dram vial with cell culture media at 37° C. to follow the schematic from FIG. 28. Each gel was placed in one of the three conditions: (1) media, (2) 100 U/mL of catalase from the bovine liver, and lastly (3) 100 U/mL of catalase from the bovine liver (see FIG. 28), which followed the same ratio as the first biocompatibility study (1:80 ratio between the gel and media).

On Day 1, the media was collected, sterile filtered through a 0.22 μm filter, and stored at −20° C. until further use. For the latter time points (Days 3 and 7), the media was replenished in the same vial with the appropriate media, the gels labelled with condition 1 (C1) were replaced with media, for condition 2 (C2), the media was replenished with 100 U/mL of Catalase, and the last condition (C3) was replenished with media. The media was removed on Day 3, where the media was stored similarly to Day 1 (the collected media was filtered through a 0.22 μm Filter and stored at −20° C. until further use). On Day 7, the media was collected, filtered, and stored at −20° C. until further use. There were three gels performed in each condition, and the experiment was repeated three times.

### 3rd Biocompatibility Study

Additional experiments were completed to determine the toxic concentration of hydrogen peroxide generated during crosslinking of the hydrogel system and viable ascorbic acid concentrations. To prepare for this experiment, specific concentrations of H₂O₂ and ascorbic acid were prepared by testing a range of concentrations and incubating at 37° C. On the following day, 200 μL of each condition was treated to pre-seeded primary chondrocytes at passage 6 and evaluated for its metabolic activity as per below. Each condition was performed in triplicate.

In another study, 1% Penicillin Streptomycin was added to cell culture media to reduce potential contamination for long-term culture. This media was added to pre-seeded primary chondrocytes at passage 7 and evaluated for its metabolic activity. Each condition was performed in triplicate.

### Chondrocyte Culture Condition

Cryopreserved Human Chondrocytes (HCH) were purchased from PromoCell at passage 2 (P2). HCH were cultured in proprietary chondrocyte growth medium and supplemented with 10% Growth Medium Supplement by PromoCell at 37° C. and 5% CO₂. The medium was changed every 2-3 days until the cells achieved 80% confluence. Once the cells reached confluence, the chondrocytes were treated with the DetachKit (purchased by PromoCell). During biocompatibility studies, cells were used at passage 7 and seeded on a 96 well-tissue culture polystyrene (TCPS, Sarstedt) plate at a cell density of 30,000 cells/mL/well. The cells were incubated in a 5% CO₂ incubator for 24 hours to ensure that they had attached to the well. The media was removed and replenished with 200 μL of the appropriate control. Similarly, 200 μL of leached media was added. All conditions were conducted in triplicates. The negative and positive controls were untreated media and 1% Triton, respectively.

### Metabolic Activity

Cellular metabolic activity was assessed by a water-soluble tetrazolium-1 assay (WST-1, Roche) [A. V. Peskin and C. C. Winterbourn, “A microtiter plate assay for superoxide dismutase using a water-soluble tetrazolium salt (WST-1),” Clinica chimica acta, vol. 293, no. 1-2, pp. 157-166, 2000; J. E. McBane, K. Cai, R. S. Labow, and J. P. Santerre, “Co-culturing monocytes with smooth muscle cells improves cell distribution within a degradable polyurethane scaffold and reduces inflammatory cytokines,” Acta Biomater, vol. 8, no. 2, pp. 488-501, February 2012]. Following the 24-hour exposure of the leachable material and controls, the treated media was aspirated and 110 μL of a 10% solution (v/v) of WST-1 reagent in media was added to each well and incubated for two hours to ensure that all viable cells converted the tetrazolium salt to formazan. The absorbance of formazan was read on a Plate Reader (Perkin Elmer) at 450 nm. The absorbance values of all treatments and the positive control were normalized to cells treated with media.

### Hydrogen Peroxide Assay

The generation of hydrogen peroxide in cell culture media was quantified by the Ferrous Oxidation-Xylenol (FOX) Assay (Pierce™ Quantitative Peroxide Assay Kit, Thermo Scientific). In short, hydrogen peroxide reacts with sorbitol to form hydroperoxyl radicals which enhance the oxidation of Fe²⁺ to Fe³⁺, further yielding a complex with xylenol orange [S. P. Wolff, “[18] Ferrous ion oxidation in presence of ferric ion indicator xylenol orange for measurement of hydroperoxides,” Methods in enzymology, vol. 233, pp. 182-189, 1994]. The leached media from the 1^{st} Biocompatibility Study was used to determine hydrogen peroxide production at Days 1, 3, 7, 10, 13, 19, and 21. 20 μL of the extracted media at the respective days was added to 200 μL of the FOX Reagent on a 96 well plate. The remaining media was completely removed and replenished with fresh media. The calibration curve was prepared by diluting a 30 wt. % hydrogen peroxide solution (Fisher Chemical) with cell culture media to concentrations between 1-100 μM as shown in FIG. 29. The absorbance was measured on a Plate Reader at 570 nm. All values were normalized to the media alone.

### Results of Biocompatibility Studies

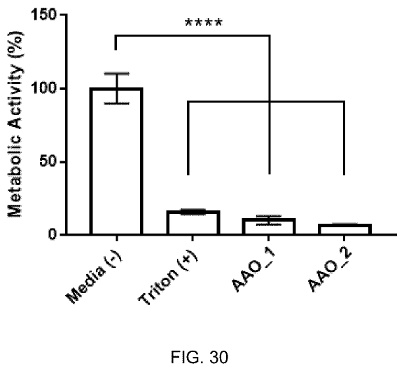

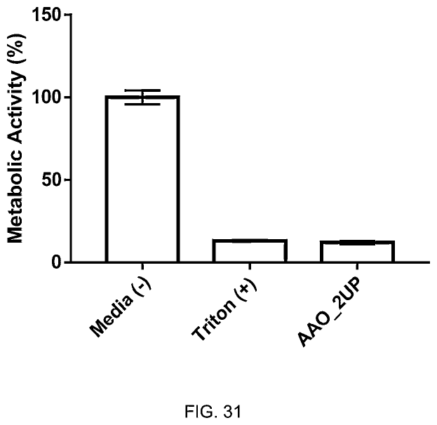

The leachable content from AAO_1 and AAO_2 was evaluated for its biocompatibility, as shown in FIG. 30. The metabolic activity from the adhesives had similar values to the positive control as there was no statistical significance. Similar biocompatibility results were observed for the unpurified adhesive (results in FIG. 31, in which the toxicity may have been attributed in part to the water-soluble agents like residual monomers and/or solvents. However, since toxicity remained in the purified product, it was believed that the toxicity associated with the purified oligomers was attributed to something associated with the polymer backbone.

Considering all the monomers in the backbone, the dopamine chemistry has established toxicity concerns resulting from the crosslinking mechanism [H. Meng, Y. Li, M. Faust, S. Konst, and B. P. Lee, “Hydrogen peroxide generation and biocompatibility of hydrogel-bound mussel adhesive moiety,” Acta Biomater, vol. 17, pp. 160-9, April 2015]. In the absence of a base, oxidant, or chelating agent, a catechol group will undergo an autoxidation process, where hydrogen peroxide is produced as a by-product. The FOX assay confirmed hydrogen peroxide in the cell culture media, as shown in Table 8, where concentrations above 100 μM were detected.

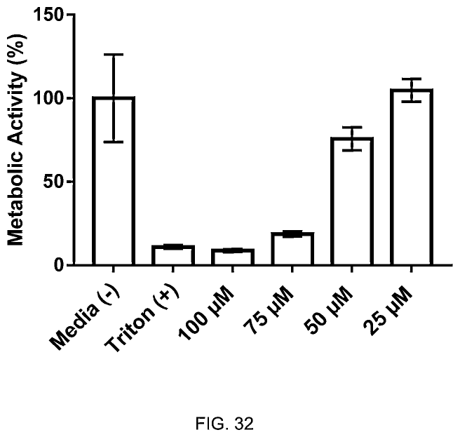

A toxicity response study was conducted to determine viable levels of H₂O₂ when incubated with primary chondrocytes. As observed in FIG. 32, concentrations below 50 μM resulted in metabolically active cells since there was no statistical significance at 50 and 25 μM of H₂O₂ to media. This concentration is beneficial for primary chondrocytes as it promotes the biosynthesis of HA, participates in intracellular signaling to maintain cartilage homeostasis, and produces appropriate levels of cytokines [P. Lepetsos and A. G. Papavassiliou, “ROS/oxidative stress signaling in osteoarthritis,” Biochim Biophys Acta, vol. 1862, no. 4, pp. 576-591, April 2016; N. Hutadilok, M. M. Smith, and P. Ghosh, “Effects of hydrogen peroxide on the metabolism of human rheumatoid and osteoarthritic synovial fibroblasts in vitro,” Ann Rheum Dis, vol. 50, no. 4, pp. 219-26, April 1991]. Though, concentrations above 50 μM evidenced cell toxicity.

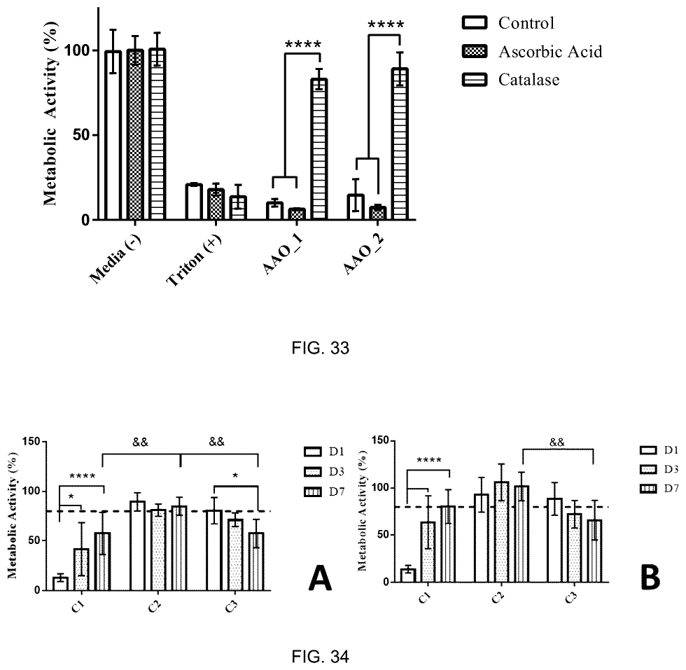

Two strategies were tested to minimize hydrogen peroxide production by incorporating 1) 100 U/mL of catalase or 2) 200 μM of ascorbic acid into the cell culture media with the phase-separated gels. After 24 hours of incubation, hydrogen peroxide concentrations depleted when catalase was added, as observed in Table 8. While the concentration of ascorbic acid (vitamin-C) in the current application did not change the outcome, it is predicted that increasing the concentration is a viable solution to reduce hydrogen peroxide further based on previous studies [[Z. Chang, L. Huo, P. Li, Y. Wu, and P. Zhang, “Ascorbic acid provides protection for human chondrocytes against oxidative stress,” Mol Med Rep, vol. 12, no. 5, pp. 7086-92, November 2015]. The addition of 100 U/mL of catalase to the cell culture media was statistically significant when observing the metabolic activity in comparison to media and the addition of 200 μM ascorbic acid. FIG. 33 supports the FOX Assay results from Table 8, confirming that the toxicity was attributed to hydrogen peroxide. There was no statistical significance between the AAO gels supplemented with catalase compared to the negative control of media supplemented with catalase.

TABLE 8
The concentration of hydrogen peroxide that leached from AAO_1
and AAO_2 at a ratio of 1:80 with respect to the cell culture media.
Hydrogen peroxide production was measured using the FOX Assay via
the Pierce ™ Quantitative Peroxide Assay Kit by Thermo
Scientific. The calibration curve is shown in FIG. 29. Standard
deviation is calculated according to the results from the
replicates performed for each condition (N = 3, n = 3).

| | Concentrations of Hydrogen Peroxide (μM) | | |
|---|---|---|---|
| | | Media + Ascorbic | Media + Catalase |
| | Media | Acid (200 μM) | (100 U/mL) |
| AAO_1 | >100 | >100 | 0 |
| AAO_2 | >100 | >100 | 0 |

While the above cell viability experiments evaluated the extracts following a one-day incubation with the gels, the subsequent experiment sought to investigate the time course of the hydrogen peroxide effect on the chondrocytes.

Based on the experimental configuration described in FIG. 28, two culture conditions were referred to as C1 and C2, where media and media plus 100 U/mL of catalase were prepared, respectively. There was also a third condition, C3, where catalase was included on the first day and then switched to media for subsequent time points. The latter group was set up to determine if traces of catalase could absorb onto the gel to inhibit hydrogen peroxide production. The results from C2 (100 U/mL of catalase in media) in FIG. 34A (AAO_1 data) and FIG. 34B (AAO_2 data) showed that the metabolic activity exceeded 80% at all time points. From FIG. 34, both oligomers at C1 Day 1 revealed the lowest metabolic activity, similar to the first biocompatibility study reported above in FIG. 30.

Validated previously from Section 3.3, the oxidation of catechol to dopamine-quinone (featured in FIG. 20) was demonstrated by a sharp increase in the absorbance at 395 nm as early as 11.5 hours, producing hydrogen peroxide as a by-product. At latter time points, there was a gradual increase in the absorbance, suggesting a lower hydrogen peroxide production. Therefore, on Days 3 and 7, a considerable increase was observed in the metabolic activity for both gels at C1, reaching statistical significance. In C3, without the presence of catalase, there was a decrease in metabolic activity at Days 3 and 7, suggesting the oxidation of catechols still produced toxic concentrations of hydrogen peroxide. This suggested insufficient catalase adhered to the gel, otherwise, the metabolic activity would follow the results from C2. In summary, there are established concerns with the production of hydrogen peroxide on Day 1, while on Day 3 and 7, the metabolic activity increased, however, the levels remained cytotoxic, according to the ISO 10993-5.

Most dopamine-based biomaterials have demonstrated successful in vivo compatibility due to the addition of an oxidant or chelating agent in which hydrogen peroxide production is minimal [J. H. Ryu, S. Hong, and H. Lee, “Bio-inspired adhesive catechol-conjugated chitosan for biomedical applications: A mini review,” Acta biomaterialia, vol. 27, pp. 101-115, 2015; M. K. Park, M.-X. Li, I. Yeo, J. Jung, B.-I. Yoon, and Y. K. Joung, “Balanced adhesion and cohesion of chitosan matrices by conjugation and oxidation of catechol for high-performance surgical adhesives,” Carbohydrate Polymers, vol. 248, p. 116760, 2020].

Hydrogen peroxide is an example of a reactive oxygen species that can amplify the production of pro-inflammatory markers. A strategy to mitigate this challenge includes adding antioxidants, including Vitamin E or selenium, to the gel system [K. M. Surapaneni and G. Venkataramana, “Status of lipid peroxidation, glutathione, ascorbic acid, vitamin E and antioxidant enzymes in patients with osteoarthritis,” Indian J Med Sci, vol. 61, no. 1, pp. 9-14, January 2007; A. Mehmood, N. Wajid, M. Rauf, S. N. Khan, and S. Riazuddin, “Vitamin E protects chondrocytes against hydrogen peroxide-induced oxidative stress in vitro,” Inflammation Research, vol. 62, no. 8, pp. 781-789, 2013]. Interestingly, the polymerized form of dopamine, polydopamine is biocompatible and can scavenge radicals [X. Liu, J. Cao, H. Li, J. Li, Q. Jin, K. Ren, and J. Ji, “Mussel-inspired polydopamine: a biocompatible and ultrastable coating for nanoparticles in vivo,” ACS Nano, vol. 7, no. 10, pp. 9384-95, Oct. 22 2013; N. Sahiner, S. Sagbas, M. Sahiner, D. A. Blake, and W. F. Reed, “Polydopamine particles as nontoxic, blood compatible, antioxidant and drug delivery materials,” Colloids Surf B Biointerfaces, vol. 172, pp. 618-626, Dec. 1 2018; X. Bao, J. Zhao, J. Sun, M. Hu, and X. Yang, “Polydopamine Nanoparticles as Efficient Scavengers for Reactive Oxygen Species in Periodontal Disease,” ACS Nano, vol. 12, no. 9, pp. 8882-8892, Sep. 25 2018]. Thus, one strategy is to include these free-radical scavengers into the gel to form a dual drug delivery system. While the encapsulated therapeutic compound (e.g. an NSAID) would gradually release, the free radical scavenger is added to minimize the hydrogen peroxide from the gel and additionally other free radicals at the target treatment site.

### Example 5: Drug Delivery

### Sample Preparation

The drug release samples were prepared by dissolving AAO_1 and AAO_2 at 20 wt. % in PBS. Additionally, 10 wt. % of celecoxib with respect to the polymer was added to the precursor to stir overnight to ensure complete homogeneity. The release media for the drug release study was prepared beforehand following a protocol by Bertram et al., as the paper identified the ion concentration of simulated synovial fluid in normal and OA patients [K. L. Bertram, U. Banderali, P. Tailor, and R. J. Krawetz, “Ion channel expression and function in normal and osteoarthritic human synovial fluid progenitor cells,” Channels (Austin), vol. 10, no. 2, pp. 148-57, 2016]. The osteoarthritic ion concentration of SSF was chosen with the addition of 0.2% Tween™ 80 to increase the solubility of celecoxib in the solution [A. Petit, M. Sandker, B. Muller, R. Meyboom, P. van Midwoud, P. Bruin, E. M. Redout, M. Versluijs-Helder, C. H. van der Lest, S. J. Buwalda, L. G. de Leede, T. Vermonden, R. J. Kok, H. Weinans, and W. E. Hennink, “Release behavior and intra-articular biocompatibility of celecoxib-loaded acetyl-capped PCLA-PEG-PCLA thermogels,” Biomaterials, vol. 35, no. 27, pp. 7919-28, September 2014; A. Dolenc, J. Kristl, S. Baumgartner, and O. Planinsek, “Advantages of celecoxib nanosuspension formulation and transformation into tablets,” Int J Pharm, vol. 376, no. 1-2, pp. 204-12, Jul. 6 2009].

To calculate the approximate drug loading, the composition was transferred into a syringe with an 18 G needle and injected into a pre-weighed vial with 1 mL of the prepared SSF at 37° C. Immediately following, the release media was removed, and the vial was weighed to determine the mass of the gel. Next, 1 mL of methanol was added to the vial to solubilize the gel and was left on a shaker overnight. The subsequent day, the sample was diluted with the 50:50 composition of MeOH/SSF to 1 ug/mL, and filtered with a 0.45 μM PTFE Membrane (Gelman Laboratory) (N=2, n=2). Similar to the drug loading calculation, the drug release experiment was conducted following a similar sample preparation as described above. Post-injection, a pre-weighed vial with 1 mL of SSF was weighed with the gel to determine the initial mass of the gel at time zero. At pre-determined time points, the fluid was removed and replenished with SSF. At each time point, the release media was diluted with a 50:50 composition of MeOH/SSF to lie within the calibration curve limits (see the Section below). Before analyses, all drug release samples were filtered with a 0.45 μM polytetrafluoroethylene membrane (Gelman Laboratory).

### Ultra Performance Liquid Chromatography

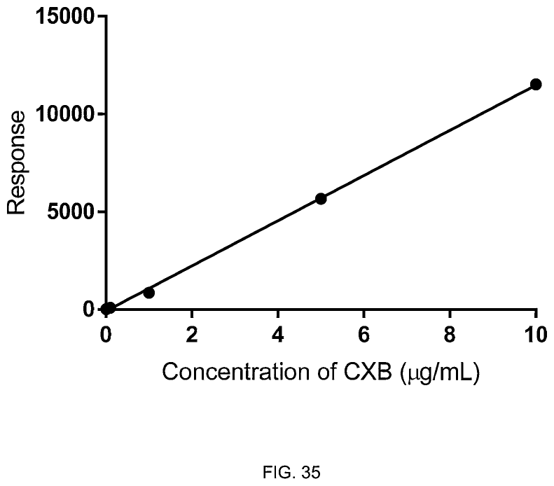

The concentration of celecoxib (CXB) was quantified using an Ultra-Performance Liquid Chromatography (UPLC) system with Acquity BEH C18 column (Waters). Celecoxib was detected by a photodiode array absorbance of 254 nm and an Acquity H-Class LC System connected to a Xevo G2-XS QTof operating in positive ion mode [M. Marashdeh, C. Stewart, A. Kishen, C. Levesque, and Y. Finer, “Drug-Silica Co-Assembled Particles Improve Antimicrobial Properties of Endodontic Sealers,” Journal of Endodontics, 2021]. The analyses were completed on Quanlynx software version 4.1. A protocol from Zheng et al. was used to quantify celecoxib [X. Zheng, J. Wen, T. H. Liu, Q. G. Ou-Yang, J. P. Cai, and H. Y. Zhou, “Genistein Exposure Interferes with Pharmacokinetics of Celecoxib in SD Male Rats by UPLC-MS/MS,” Biochem Res Int, vol. 2017, p. 6510232, 2017]. The mobile phases used to detect the drug were (1) 0.1% formic acid and (2) acetonitrile (ACN). The total run time per sample was 2.5 minutes utilizing a gradient process. During the 0-to-0.5-minute period, a 40:60 of 0.1% formic acid: ACN was chosen, following a linear increase of ACN to 95% at 1.5 minutes, and lastly, ACN was decreased to 60% at the second minute. The flow rate remained constant at 0.4 ml/min. A calibration curve was prepared covering the range of 0.025 to 10 μM, diluted with 50:50 of MeOH/SSF as shown in FIG. 35.

### Results of Drug Delivery Studies

The phase-separated gels (AAO_1 and AAO_2 loaded with 10 wt. % CXB) were formed in a SSF at 37° C., where the release media was immediately removed and replaced with methanol to determine the total loading within the gel. Summarized in Table 9 is the theoretical value added to the vehicle compared with the actual concentration of CXB measured by the UPLC.

TABLE 9
The theoretical and actual concentration of CXB loaded into the
phase-separated gels (AAO_1 and AAO_2). The theoretical
value is the measured mass of CXB as a function of the oligomer,
while the actual concentration was obtained from the UPLC results.
The standard deviation is for four independent gels per oligomer.

The Concentration of CXB as a
Function of the Vehicle

| | AAO_1 | AAO_2 |
|---|---|---|
| Theoretical (potential total | ~10% | ~10% |
| CXB concentration) | | |
| Actual (amount encapsulated, | ~5.6 ± 0.4% | ~5.4 ± 0.2% |
| n = 4/group) | | |

The results from Table 9 indicated that the gel process was successful in encapsulating a little over 50% of the total available drug, resulting in a maximum loading of 5.5 wt. % of CXB. Without wishing to be bound by a theory, a potential reason for the lower loading capacities is that the two oligomers were completely saturated with celecoxib, based on their hydrophilic character. When comparing these oligomers to a PCLA-PEG-PCLA oligomer system, 10 wt. % of CXB was successfully encapsulated, however, these materials have a greater degree of hydrophobicity within the oligomer backbone [D. A. Prince, I. J. Villamagna, C. C. Hopkins, J. R. de Bruyn, and E. R. Gillies, “Effect of drug loading on the properties of temperature-responsive polyester-poly (ethylene glycol)-polyester hydrogels,” Polymer International, vol. 68, no. 6, pp. 1074-1083, 2019]. Since AAO_1 and AAO_2 comprise of more hydrophilic domains than hydrophobic ones, a lower loading capacity may be anticipated. One strategy for increasing the CXB loading is by including PCL-PEG-PCL oligomers into the gel.

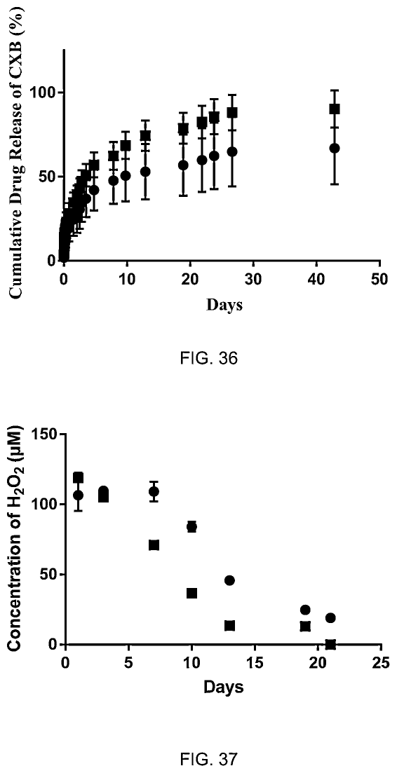

The actual loaded mass of CXB from Table 9 was denoted as 100% cumulative release, shown in FIG. 36. The cumulative release was measured for 42 days for both oligomers, with cumulative release values of 67% and 90% for AAO_1 and AAO_2, respectively.

Initially, CXB followed a burst release, in which 25% was released within the first day as observed in other thermoresponsive systems that encapsulated a hydrophobic drug [B. Miao, C. Song, and G. Ma, “Injectable thermosensitive hydrogels for intra-articular delivery of methotrexate,” Journal of Applied Polymer Science, vol. 122, no. 3, pp. 2139-2145, 2011; C. Y. Gong, S. Shi, P. W. Dong, B. Yang, X. R. Qi, G. Guo, Y. C. Gu, X. Zhao, Y. Q. Wei, and Z. Y. Qian, “Biodegradable in situ gel-forming controlled drug delivery system based on thermosensitive PCL-PEG-PCL hydrogel: part 1—Synthesis, characterization, and acute toxicity evaluation,” J Pharm Sci, vol. 98, no. 12, pp. 4684-94, December 2009; B. Jeong, Y. H. Bae, and S. W. Kim, “Drug release from biodegradable injectable thermosensitive hydrogel of PEG-PLGA-PEG triblock copolymers,” J Control Release, vol. 63, no. 1-2, pp. 155-63, Jan. 3 2000]. This may occur due to the adsorption of the drug to the gel's surface rather than its interaction with the hydrophobic domains of PCL.

Following the burst release, two main principles dictate the release profile, which includes (1) diffusion from the gel's pores or (2) diffusion following the erosion/degradation of the polymer backbone [X. Huang and C. S. Brazel, “On the importance and mechanisms of burst release in matrix-controlled drug delivery systems,” J Control Release, vol. 73, no. 2-3, pp. 121-36, Jun. 15 2001; M. Qiao, D. Chen, X. Ma, and Y. Liu, “Injectable biodegradable temperature-responsive PLGA-PEG-PLGA copolymers: synthesis and effect of copolymer composition on the drug release from the copolymer-based hydrogels,” Int J Pharm, vol. 294, no. 1-2, pp. 103-12, Apr. 27 2005; S. Dash, P. N. Murthy, L. Nath, and P. Chowdhury, “Kinetic modeling on drug release from controlled drug delivery systems,” Acta Pol Pharm, vol. 67, no. 3, pp. 217-23, May-June 2010].

Due to the slow degradation/erosion profile of PCL as validated in FIG. 24 and the literature [H. Sun, L. Mei, C. Song, X. Cui, and P. Wang, “The in vivo degradation, absorption and excretion of PCL-based implant,” Biomaterials, vol. 27, no. 9, pp. 1735-1740, 2006; C. X. Lam, D. W. Hutmacher, J. T. Schantz, M. A. Woodruff, and S. H. Teoh, “Evaluation of polycaprolactone scaffold degradation for 6 months in vitro and in vivo,” Journal of Biomedical Materials Research Part A: An Official Journal of The Society for Biomaterials, The Japanese Society for Biomaterials, and The Australian Society for Biomaterials and the Korean Society for Biomaterials, vol. 90, no. 3, pp. 906-919, 2009], diffusion of CXB from the gel's pores is the main mechanism that dictates the release profile. This provides evidence that the two oligomer systems comprise of a sufficient pore size by which the hydrophobic drug can diffuse out from the gel despite the presence of chemical crosslinking. This was confirmed as a small molecular weight molecule such as hydrogen peroxide was detected, as shown in FIG. 37.

When considering the therapeutic response of CXB for the application of OA, it was observed from in vitro experiments that concentrations as low as 0.38 μg/mL demonstrated a reduction of PGE₂ levels and an increase in proteoglycan synthesis evaluated from human OA cartilage [S. C. Mastbergen, N. W. Jansen, J. W. Bijlsma, and F. P. Lafeber, “Differential direct effects of cyclo-oxygenase-1/2 inhibition on proteoglycan turnover of human osteoarthritic cartilage: an in vitro study,” Arthritis Res Ther, vol. 8, no. 1, p. R2, 2006].

In previous work reported with the use of PCLA-PEG-PCLA oligomer, a maximum concentration of 270 μg/mL was detected in the synovial fluid of an equine where no inflammation was observed [D. A. Prince, I. J. Villamagna, A. Borecki, F. Beier, J. R. de Bruyn, M. Hurtig, and E. R. Gillies, “Thermoresponsive and Covalently Cross-Linkable Hydrogels for Intra-Articular Drug Delivery,” ACS Applied Bio Materials, vol. 2, no. 8, pp. 3498-3507, 2019]. Thus, the concentration of CXB loaded into the oligomer system can provide a therapeutic effect since all measured concentrations of CXB were greater than 3.0 μg/mL while the highest concentration detected during one-time point did not exceed 57 μg/mL.

AAO_1 and AAO_2 exhibited a cumulative release of 67% and 90%, respectively, over 42 days. Providing a secondary barrier for the drug to be encapsulated (e.g. nanoparticle loaded drug) or the inclusion of additives such as polyvinyl alcohol (PVA) are two strategies that could further delay the release of a hydrophobic drug.

As can be seen therefore, the examples described above and illustrated are intended to be exemplary only. The scope is indicated by the appended claims.

1. An amphiphilic oligomer having a formula
(D-C-A-C)ₙ-B
or
(D-C—B—C)ₙ-A
wherein
n=2 or 3;
D is a terminal adhesive group;
A is a hydrophilic segment having a molecular weight between 600 and 2000;
B is a degradable hydrophobic segment having a molecular weight between 900 and 2000; and
C is a linking group comprising a urethane or urea linkage.

2. The amphiphilic oligomer of claim 1, wherein the ratio of hydrophobic segment to hydrophilic segment is between 0.3 and 2.1.

3. The amphiphilic oligomer of claim 1 wherein the hydrophobic segment has a molecular weight between 800 and 1400.

4. The amphiphilic oligomer of claim 1, wherein the hydrophilic segment has a molecular weight between 800 and 1200.

5. The amphiphilic oligomer of claim 1, wherein the molecular weight of the amphiphilic oligomer is between 4400 and 10,000.

6. The amphiphilic oligomer of claim 1 having the formula (D-C-A-C)ₙ—B.

7. The amphiphilic oligomer of claim 1, wherein the hydrophobic segment is derived from a polyester, including polycaprolactone (PCL), polylactic acid, polyglycolic acid, polyamide, polyurethane (PU), cellulosic oligomer, oligosaccharide, poly(alkenedicarboxylate) poly(hydroxybutyrate), poly anhydrides, poly peptides, poly(β-hydroxyalcanoate), poly(hydroxybutyrate-co-hydroxyvalerate) or poly(p-dioxanone).

8. The amphiphilic oligomer of claim 7, wherein the hydrophilic segment is derived from polyethylene glycol (PEG), polyvinyl alcohol (PVA), polyvinylpyrrolidone (PVP), polyethylene oxide (PEO), co-poly(ethylene oxide)-b-poly(propylene oxide), poly(hydroxyethylmethacrylate) (polyHEMA), poly acrylic acid or poly acrylic acid mono-salts (H⁺ substituted for Na⁺, K⁺).

9. The amphiphilic oligomer of claim 1, wherein the terminal adhesive group is a terminal benzene-1,2-diol derivative or a terminal adhesive benzene-1,2,3-triol.

10. The amphiphilic oligomer according to claim 1, wherein D has a structure selected from: 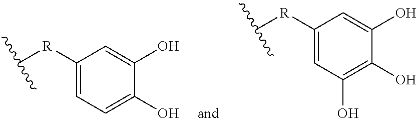
wherein R is present or absent and when present is a C1-C6 alkyl group or C1-C6 alkene optionally substituted with OH or NH₂.

11. A self-assembled hydrogel comprising one or more oligomer species as defined in claim 1.

12. The self-assembled hydrogel of claim 11 formed from a single amphiphilic oligomer species of the formula (D-C-A-C)ₙ—B.

13. The self-assembled hydrogel of claim 11 formed from a first amphiphilic oligomer of the formula (D-C-A-C)ₙ—B, wherein A is derived from PEG and has a molecular weight of about 1000, B is derived from PCL and has a molecular weight of about 1250 and n=2; and
a second amphiphilic oligomer of the formula (D-C-A-C)ₙ—B, wherein A is derived from PEG and has a molecular weight of about 1000, B is derived from PCL and has a molecular weight of about 900 and n=3.

14. A method of preparing a gel polymer matrix comprising dissolving an amphiphilic oligomer of claim 1 in an aqueous solvent and raising the temperature of the solution to a gelation temperature of the gel polymer matrix.

15-22. (canceled)

23. A process of preparing an amphiphilic oligomer comprising:
A) reacting one of
a hydrophilic polyol (A′) having a molecular weight between 600 and 2000 and
a hydrophobic polyol (B′) having a molecular weight between 900 and 2000
with a diisocyanate under conditions and for a time sufficient
to obtain a compound of the formula
C′-A-C′ or C′—B—C′
wherein A and B are as defined in claim 1, and C′ comprises a urethane or urea linkage and an isocyanate group;
B) reacting the reaction product of step A with
a hydrophilic polyol having a molecular weight between 600 and 2000 or
a hydrophobic polyol having a molecular weight between 900 and 2000;
wherein if a hydrophilic polyol is reacted in step (A), a hydrophobic polyol is reacted in step (B) and if a hydrophobic polyol is reacted in step (A), a hydrophilic polyol is reacted in step (B); and
C) reacting the reaction product of step B with a 4-alkylbenzene-1,2-diol derivative or a 5-alkylbenzene-1,2,3-triol derivative selected from the group consisting of:

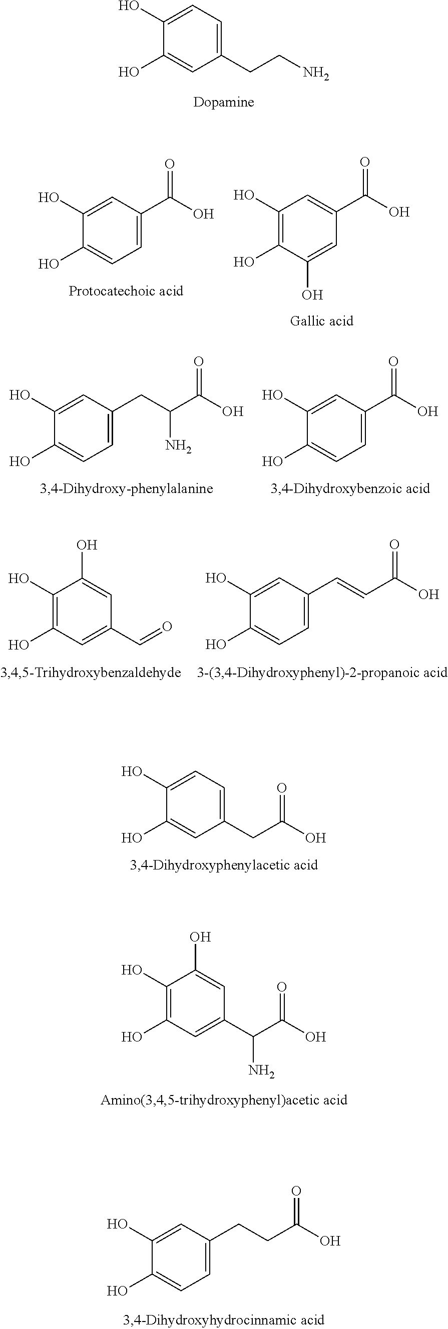

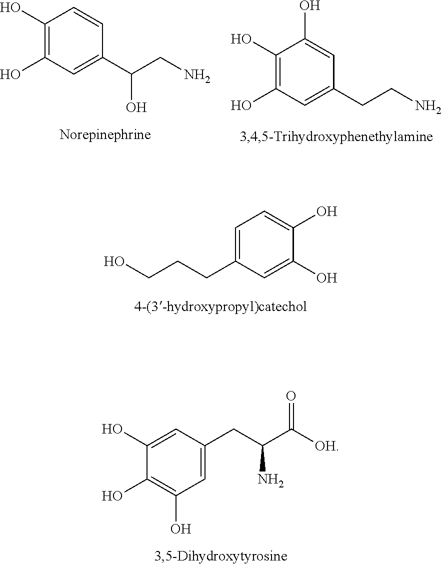 [SMILES: NCC(O)c1ccc(O)c(O)c1 ; NCCc1cc(O)c(O)c(O)c1 ; OCCCc1ccc(O)c(O)c1 ; N[C@@H](Cc1cc(O)c(O)c(O)c1)C(=O)O]

24. The process of claim 23, wherein the diisocynate molecule is selected from:

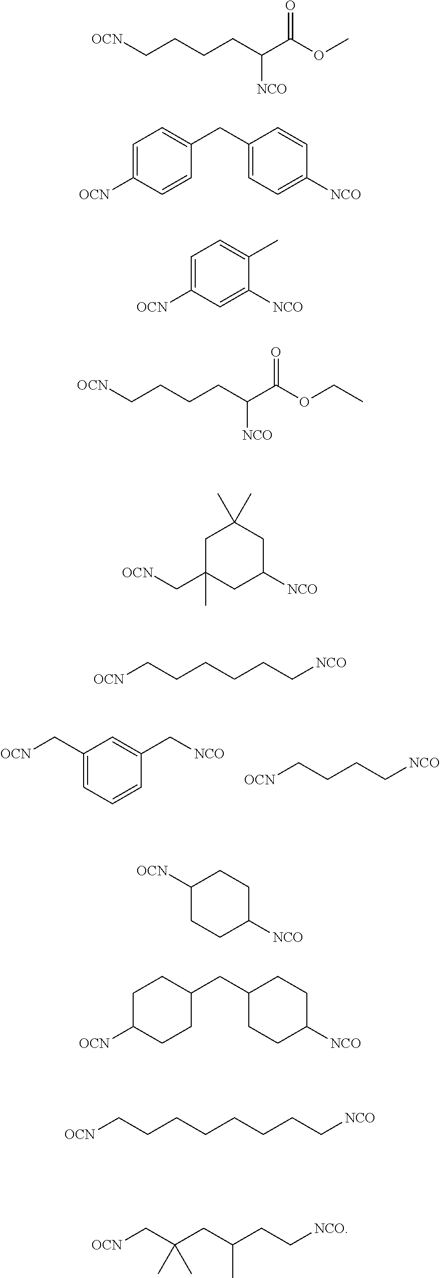

25-33. (canceled)

34. The amphiphilic oligomer of claim 9, wherein the hydrophobic segment is derived from polycaprolactone and the hydrophilic segment is derived from PEG.

35. The self-assembled hydrogel of claim 11, wherein the hydrophobic segment is derived from polycaprolactone and the hydrophilic segment is derived from PEG

36. The self-assembled hydrogel of claim 13, wherein the self-assembled hydrogel comprises between 40 and 60 wt % of the first amphiphilic oligomer and between 40 and 60 wt % of the second amphiphilic oligomer, based on the combined weight of the amphiphilic oligomers.

37. The process of claim 23, wherein the hydrophobic polyol is polycaprolactone (PCL), polylactic acid, polyglycolic acid, a polycarbonate, polyamide, polyurethane (PU), cellulosic oligomer, oligosaccharide, poly(alkenedicarboxylate), poly(hydroxybutyrate), poly anhydrides, poly peptides, poly(p-hydroxyalcanoate), poly(hydroxybutyrate-co-hydroxyvalerate) or poly(p-dioxanone); and wherein the hydrophilic polyol is polyethylene glycol (PEG), polyvinyl alcohol (PVA), polyvinylpyrrolidone (PVP), polyethylene oxide (PEO), co-poly(ethylene oxide)-b-poly(propylene oxide), poly(hydroxyethylmethacrylate) (polyHEMA), poly acrylic acid or poly acrylic acid mono-salts (H⁺ substituted for Na⁺, K⁺).
